(19) United States
(12) Patent Application Publication
(10) Pub. No.: US 2018/0105793 A1
(43) Pub. Date: Apr. 19, 2018

(54) DEVICE WITH MULTIPLE MICROENVIRONMENTS AND METHODS THEREOF

(71) Applicants: Amit Pathak, Clayton, MO (US); Samila Nasrollahi, St. Louis, MO (US); Bapi Sarker, St. Louis, MO (US)

(72) Inventors: Amit Pathak, Clayton, MO (US); Samila Nasrollahi, St. Louis, MO (US); Bapi Sarker, St. Louis, MO (US)

(73) Assignee: WASHINGTON UNIVERSITY, St. Louis, MO (US)

(21) Appl. No.: 15/730,371

(22) Filed: Oct. 11, 2017

Related U.S. Application Data
(60) Provisional application No. 62/409,198, filed on Oct. 17, 2016.

(51) Int. Cl. C12N 5/00; C12M 3/00; C12M 3/06; C12M 1/12

(52) U.S. Cl. CPC C12N 5/0075; C12M 3/006; C12N 2502/30; C12M 25/06; C12M 23/16

(57) ABSTRACT

Among the various aspects of the present disclosure is the provision of a device with multiple microenvironments and methods of use and manufacture thereof. An aspect of the present disclosure provides for a device for evaluating cell invasion. Another aspect provided by the present disclosure includes a method of making a device for evaluating cell invasion. Another aspect to the present disclosure provides for a method of testing a drug in vitro. Another aspect of the present disclosure provides for a method of identifying targets.

### CROSS-REFERENCE TO RELATED APPLICATIONS

This application claims priority from U.S. Provisional Application Ser. No. 62/409,198 filed on 17 Oct. 2016, which is incorporated herein by reference in its entirety.

### STATEMENT REGARDING FEDERALLY SPONSORED RESEARCH OR DEVELOPMENT

This invention was made with government support under grant number CMM11454016 awarded by the National Science Foundation. The government has certain rights in the invention.

### FIELD OF THE INVENTION

The present disclosure generally relates to devices with multiple stiffness microenvironments of the cancer invasion trajectory for the testing of cancer related drugs.

### BACKGROUND OF THE INVENTION

The aggressive invasion of cancer cells into far reaches of the body is a hallmark of metastasis and is widely considered to be a key obstacle to the success of emerging therapies. To form metastases in distant organs, cancer cells undergo a stepwise process, which includes detachment from the primary tumor, escape through the surrounding matrix, intravasation to blood vessels, dissemination through circulation, extravasation to a secondary tissue site, and repopulation of secondary tumors. Throughout these steps, cancer cells successfully adapt to foreign microenvironments through a property known as ‘cellular plasticity’. The ability of cancer cells to alter their phenotypic and morphological characteristics in wide-ranging microenvironments allows persistent tumor invasion and renders the metastasis trajectory highly unpredictable.

As cells pass through a tissue microenvironment, a distinct set of mechanosensitive signaling events occur, such as clustering of integrin-based adhesion proteins into focal adhesions, Rho-ROCK activation, and nuclear localization of transcriptional regulators YAP and SNAIL1. Recently, mechanical dosing of human mesenchymal stem cells on matrices of tunable stiffness has been found to regulate mechanical memory-dependent lineage commitment decisions, and this process is shown to depend on YAP activity. To study cell migration on heterogeneous matrices, gel systems with gradient stiffness have been used to show durotaxis for single and collective cells, and define specific roles of myosin isoforms in cell polarization during spontaneous migration across these substrates. However, it remained unknown whether the cells that are primed on a given ECM for a defined duration retain their mechanosensitive signatures even after moving to a new microenvironment.

Mechanical properties of the extracellular matrix (ECM) influence phenotypic and genotypic cellular responses, which regulate cell differentiation, migration, and proliferation. In particular, matrix stiffness regulates cellular forces, adhesions, protrusions, and polarization through mechanotransductive signaling, all of which in turn lead to mechanosensitive variations in both single and collective cell migration phenotypes. In reality, migratory cells do not continually reside in just one type of matrix. Instead, cells singly and collectively migrate through mechanically heterogeneous matrices, forming the basis of fundamental biological processes including embryonic development, wound healing, regeneration, and cancer metastasis. In cancer metastasis, the mechanical properties of the primary tumor microenvironment are known to induce de-clustering and outward migration of cancer cells into the surrounding tissue, which represent the first steps of tumor invasion. As a result, cancer cells invade through the stromal matrix, intravasate into blood vessels, disseminate through circulation, extravasate to a secondary tissue site, and reactivate their growth in other organs.

Throughout these steps, cells adapt to tissue microenvironments of differing mechanical properties. As cells pass through a tissue microenvironment a distinct set of mechanosensitive signaling events occur. However, it remains unknown whether these ECM-regulated cellular signatures persist after cells migrate into a new microenvironment, and if so how this influence cell migration.

For example, cancer stem cells escape a primary tumor by undergoing epithelial-to-mesenchymal transition (EMT) and then integrate with secondary tissue sites by transitioning back to an epithelial phenotype through a mesenchymal-to-epithelial transition (MET). Cellular plasticity in motile cells is manifested by variable modes of migration, e.g., mesenchymal, amoeboid, and collective, depending on the surrounding microenvironment. During these processes, varied microenvironments encountered by cancer cells in turn modify the state of those cells. This ‘reciprocal plasticity’ of both the cancer cells and the tissue microenvironments not only generates diverse routes for metastasis progression, but also gives rise to tumor heterogeneity in which functional states of cancer cells within a given tumor can vary significantly. Taken together, the unpredictable trajectories of metastasis and the heterogeneous populations of cancer cells in a given tumor have severely stymied the development of robust therapeutic strategies. Since cellular plasticity throughout the metastasis trajectory is at the root of these issues, a systematic analysis of tumor heterogeneity is critically tied to a better understanding of time variation of microenvironment-dependent cancer cell plasticity after escaping the primary tumor.

Therefore, there is a need for a device that provides for multiple microenvironments of the cancer invasion trajectory for the testing of cancer related drugs. The device should integrate multiple steps of metastasis, from primary tumor to secondary metastatic sites, permits controlled manipulation of biomechanical properties of varied microenvironments, and allows cellular measurements at every step. Moreover, these devices mimic key features of any given microenvironment.

### SUMMARY OF THE INVENTION

Among the various aspects of the present disclosure is the provision of a device with multiple microenvironments and methods of use and manufacture thereof.

Additional embodiments and features are set forth in part in the description that follows, and will become apparent to those skilled in the art upon examination of the specification or may be learned by the practice of the disclosed subject matter. A further understanding of the nature and advantages of the disclosure may be realized by reference to the remaining portions of the specification and the drawings, which forms a part of this disclosure.

An aspect of the present disclosure provides for a device for evaluating cell invasion.

For example, the device can include a substrate material comprising at least two regions, wherein a first region has a first stiffness and a second region has a second stiffness; or a plurality of cells seeded on the first region, wherein the cells are preconditioned to the first region before migrating to the second region. As another example, the device can provide a first region mimicking a primary tumor site and the second region mimicking a secondary invasion site; the first region can have a different stiffness value than the second region; or the first region can have an increased stiffness value compared the second region. As another example, the substrate can include a third region comprising at least one microchannel, wherein the third region is located between the first region and the second region; or a fourth region mimicking stromal tissue, wherein the fourth region is located between the first region and the third region. As another example, the device can include at least one microchannel that is a flow channel. As another example, the device can include mammary cells. As another example, the device can include a substrate comprising polyacrylamide (PA), polydimethylsiloxane (PDMS), collagen, or fibrin, or combinations thereof.

As another example, the device can include a substrate material, wherein the substrate material in the first region is a different polymer than the substrate material in the second region.

Another aspect provided by the present disclosure includes a method of making a device for evaluating cell invasion.

For example, the method can include polymerizing a substrate comprising at least two regions, wherein a first region has a first stiffness and a second region has a second stiffness; and seeding a plurality of cells on the first region, wherein the cells are preconditioned to the first region before migrating to the second region.

As another example, the device can include a substrate, wherein at least a portion of the substrate is polymerized though photopolymerization.

As another example, the device can include a substrate, wherein the substrate comprises polyacrylamide (PA), polydimethylsiloxane (PDMS), collagen, or fibrin, or combinations thereof. As another example, the device can include a substrate, wherein the substrate further comprises a fourth region.

As another example, the method further comprises fabricating microchannels in a third region of the substrate.

As another example, the cells are initially limited to the first region to be preconditioned to the first region by placing a stencil over the second region to prevent migration to the second region until after the cells have been preconditioned.

As another example, the cells are initially limited to the first region to be preconditioned to the first region by: limiting the cells seeded onto the first region, selecting a location for seeding the cells that is a distance from the second region, or increasing the first region size, or combinations thereof.

Another aspect to the present disclosure provides for a method of testing a drug in vitro.

For example, the method can include seeding cells on a first region of a device comprising a substrate comprising at least two regions, wherein the first region has a first stiffness and a second region has a second stiffness; administering a drug to the cells on the first region or the second region; or observing cell characteristics or observing cell migration properties.

As another example, the observed cell characteristics or cell migration properties are selected from the group consisting of migration speed, migration distance, and molecular expressions, and combinations thereof.

As another example, the substrate can further comprise a third region comprising at least one microchannel, wherein the third region is located between the first region and the second region. As another example, the substrate can further comprise a fourth region mimicking stromal tissue, wherein the fourth region is located between the first region and the third region.

As another example, the cells can be primary or immortalized cancer cells, optionally, squamous carcinoma, mammary cells, breast cancer cells, mixed co-cultured cell types, or primary cells from the tumor, optionally from a human or a mammal.

Another aspect of the present disclosure provides for a method of identifying targets. For example, the method can comprise performing RNA-seq for genomic analyses to narrow down memory-related targets.

As another example, the method can further comprise disrupting a target that is identified to be implicated in memory-storing abilities; and comparing cell characteristics or invasions after inhibiting memory-related signals.

Other objects and features will be in part apparent and in part pointed out hereinafter.

### DESCRIPTION OF THE DRAWINGS

Those of skill in the art will understand that the drawings, described below, are for illustrative purposes only. The drawings are not intended to limit the scope of the present teachings in any way.

FIG. 1A-FIG. 1C illustrates cell plasticity and memory across distinct ECMs. FIG. 1A illustrates cell response to separate ECMs. FIG. 1B illustrates pure plasticity, where cells quickly adapt to the new ECM. FIG. 1C illustrates cells with memory which continue to depend on the old ECM.

FIG. 2A-FIG. 2B illustrates the fabrication of 2D substrates of heterogeneous stiffness. FIG. 2A illustrates a polyacrylamide (PA) solution mixed with a photoinitiator that is polymerized through a mask under UV exposure, resulting in a substrate with regions of dissimilar stiffness. FIG. 2B shows alternate methods for making PA gels of heterogeneous stiffness by controlled mixing of PA ratios.

FIG. 3 illustrates multiscale measurements in a table of proposed measurements of cellular properties across length scales—from single/clustered cells to subcellular molecules. Dotted lines indicate known connections among various parameters.

FIG. 4A-FIG. 4B illustrates collective migration speed due to past stiffness. (FIG. 4A) Schematic of collective migration on a PA substrate with dissimilar stiffness (Ep, or Es) regions. Migration speed “vs” is calculated after cells arrive in the secondary ECM, Es. (FIG. 4B) Cells originally seeded on stiffer primary ECM (greater Ep) migrate faster on a given secondary ECM. This difference, denoted as a memory index “μ”, increases for α-catenin knockdown MCF10A cells.

FIG. 5A-FIG. 5B illustrates fabricating PA channels of varying stiffness and confinement. (FIG. 5A) Culture of epithelial clusters in channels of defined stiffness and confinement made by polymerizing PA solutions in lithographically fabricated Si-masters. (FIG. 5B) Introduce regional variations in channel stiffness through photo-polymerization of PA.

FIG. 6A-FIG. 6B shows enhanced EMT in confinement. (FIG. 6A) E-cad localization reduces in narrower and stiffer channels. (FIG. 6B) Confocal images of E-cad expressions in soft (1 kPa) and stiff (120 kPa) PA channels of 200 & 20 μm width.

FIG. 7A-FIG. 7B illustrates fabrication of 3D degradable ECMs. (FIG. 7A) A step-wise method for polymerizing a collagen gel around a PA substrate, which results in a setup of a 2D primary ECM and a 3D degradable secondary ECM surrounding the tumor cells. (FIG. 7B) Similar step-wise polymerization could be repeated to achieve a three-gel system encapsulated with context-specific types of cells.

FIG. 8 illustrates a trajectory of 4D metastasis in a chip. Schematic of a proposed device (on the left) which includes four key steps of the metastasis trajectory—EMT at primary tumor site, invasion through the stromal tissue, circulation through channels of varying length and curvature, and finally, MET and growth at a secondary tumor site.

FIG. 9A-FIG. 9C shows migration of cell sheet across mPA substrates of dissimilar stiffness. (FIG. 9A) Schematic describing the fabrication steps of 2D substrates of heterogeneous stiffness through modular polymerization of PA solutions of distinct compositions, resulting in dissimilar ECM stiffness in adjoining primary and secondary regions. All cell measurements are conducted in the secondary ECM. (FIG. 9B) Primary ECM stiffness Pₛ regulates the leading edge migration speed of MCF-10A and (FIG. 9C) MCF-7 cells. Horizontal square brackets denote statistical significance (p<0.05). N>15 per condition from at least two separate experiments. Scale bar=50 μm.

FIG. 10A-FIG. 10B shows YAP activity depends on past ECM stiffness. Quantification of subcellular YAP localization and representative images of YAP (green) and nuclei (blue) for (FIG. 10A) MCF10A and (FIG. 10B) MCF7 cells preconditioned on either soft or stiff primary ECMs. Data (top row) represented as the percentage of cells with nuclear, cytoplasmic or intermediate (both) YAP localization, averaged from a cell population of at least 40 cells for 10 different fields of views. Horizontal square brackets denote statistical significance (p<0.05). Scale bar=50 μm.

FIG. 11A-FIG. 11C shows Cellular motions during collective migration across ECMs of dissimilar stiffness. PIV analysis of MCF10A cell monolayers migrating across mPA gels provide (FIG. 11A) correlation length and (FIG. 11B) Order parameter of the velocity vectors. (FIG. 11C) Brightfield images (top row) and heatmaps showing spatial distribution of velocity fields (middle row) and order parameter (bottom row) at a given time for varying primary stiffness. Horizontal square brackets denote statistical significance (p<0.05). N>15 per condition from at least two separate experiments. Scale bar=50 μm.

FIG. 12A-FIG. 12E shows migration and YAP activity analyses for Rho-activated cells across ECMs of heterogeneous stiffness. MCF10A-Rho-CA cells exhibit mechanical memory-dependent migration in terms of (FIG. 12A) leading edge migration speed, (FIG. 12B) correlation length, and (FIG. 12C) order parameter of the velocity vectors. (FIG. 12D, FIG. 12E) Quantification of subcellular YAP localization and representative images of YAP (green) and nuclei (blue) after preconditioning on either soft or stiff primary ECMs. Horizontal square brackets denote statistical significance (p<0.05). N>15 different monolayers from at least two experiment. Scale bar=50 μm.

FIG. 13A-FIG. 13B shows a contiguous substrate with regions of dissimilar stiffness. (FIG. 13A) Schematic describing the fabrication steps of mPA substrates of heterogeneous stiffness through modular polymerization of PA solutions of distinct compositions, resulting in dissimilar ECM stiffness in adjoining primary and secondary regions. (FIG. 13B) Atomic Force Microscopy (AFM) measurements of Young's Modulus of PA gels plotted in logarithmic scale at different locations within a substrate with dissimilar primary and secondary ECM regions. Stiffness values are averaged over 1 mm length intervals and plotted along with scattered data points and error bars (SEM). N>150. Data is included from at least 3 different PA gels, in which the left side was intended to be stiff (acrylamide/bisacrylamide=12/0.6%) and right side as soft (acrylamide/bisacrylamide=4/0.2%) matrix.

FIG. 14A-FIG. 14B shows collagen I coating on soft and stiff PA gels. Rat-tail collagen type I was labelled using Sulfo-Cyanine5 NHS ester, as described previously*. (FIG. 14A) Soft (0.5 kPa) and stiff (50 kPa) PA gels coated with 0.05 mg/ml of this labeled collagen I were imaged. Scale bar=50 μm. (FIG. 14B) The integrated density of pixel intensity measured from these collagen images shows insignificant difference in soft and stiff gels. These measurements were repeated after removing the PDMS stencil, which shows robust collagen coating with or without PDMS interaction.

FIG. 15A-FIG. 15C shows collective cell migration speed depends on priming by the past ECM stiffness. (FIG. 15A) Representative leading-edge tracks of monolayers of MCF10A cells recorded for 12 h (3 h interval) in the secondary ECM after 3-day priming, with color-coding for migration speed. Arrows indicate direction of migration. Scale bar=100 mm. (FIG. 15B) Average leading-edge migration speed for MCF10A, A431, and MCF7 cells migrating on secondary ECM (S=0.5, 50 kPa) after defined priming (P=0.5, 50 kPa). Horizontal square brackets denote statistical significance (p<0.05). N>15. (FIG. 15C) Average leading edge migration speed for MCF10A cells after 1, 2, or 3 days of priming. *p<0.05, with horizontal square brackets denoting statistical significance (p<0.05). ns=no significant difference. Error bars=SEM. N>10.

FIG. 16A-FIG. 16B shows leading-edge tracks of representative monolayers of MCF7 and A431 cells. Plots describing the leading-edge tracks of representative monolayers of (FIG. 16A) MCF7 and (FIG. 16B) A431 cells recorded for 12 hours after entering the secondary ECM; tracks color-coded based on the leading-edge migration speed. In each case, four representative leading-edge tracks at 3 h interval are plotted on soft or stiff secondary ECMs, which were previously primed on stiff or soft primary ECMs. Arrows indicate the general direction of migration. Scale bar=100 μm.

FIG. 17A-FIG. 17E shows monolayer dynamics and temporal variation of memory-dependent migration. (FIG. 17A) Heatmap showing the spatial distribution of velocity magnitude at a given time instant for MCF10A cell monolayer migration. (FIG. 17B) Position-time kymographs of velocity magnitude and order parameter obtained from PIV analysis demonstrate the time evolution of monolayer motion. Kymographs were computed by averaging the velocity magnitude and order parameter of individual velocity vectors in the x-direction over the y-coordinate for every time point. Average (FIG. 17C) correlation length and (FIG. 17D) order parameter of the velocity vectors. Horizontal square brackets denote statistical significance (p<0.05). N>15. (FIG. 17E) Plot describes the leading-edge migration speed over time, tracked for 96 h in secondary ECM after 3-day priming of cell monolayer. All bar plots are averaged over quantities measured in the first 48 h of migration (depicted by the shaded region), which corresponds to the period of maximal memory. N>10. Error bars=SEM.

FIG. 18 shows single cell trajectories within the migrating monolayer. Trajectories of single cells located at the leading edge and inside (at least 200 μm behind the leading edge) the monolayer of cells migrating on soft (top) or stiff (bottom) secondary ECM, after priming on soft or stiff primary ECMs. Tracks were color coded according to migration speed for each cell.

FIG. 19A-FIG. 19B shows alignment of single cell movement within the monolayer depends on primary ECM stiffness. Heatmap of order parameter (top row), vector field describing the direction of velocity vectors (middle row), and rose plot (bottom row) demonstrating the distribution of the angle between the instantaneous velocity vector and the x-axis, which were obtained by analyzing the trajectories of single cells for MCF-10A monolayer migrating on (FIG. 19A) soft and (FIG. 19B) stiff secondary ECM after being primed on soft or stiff primary ECMs.

FIG. 20A-FIG. 20E shows cytoskeletal machinery in memory-dependent migration. (FIG. 20A) Immunofluorescent staining of pMLC (green), F-actin (phalloidin, red), and DAPI (blue) in top-panel and paxillin (red) and DAPI (blue) in bottom-panel for MCF10A cell monolayers on the secondary ECM after 2 days of migration (post-priming). Scale bar=100 mm. Quantification of (FIG. 20B) actin fiber alignment, (FIG. 20C) normalized pMLC expression, and (FIG. 20D) FA area. N>40. (FIG. 20E) Variation of spreading area of single cells within the MCF10A cell monolayer relative to the distance from the leading edge showing stiffness-independent cell spreading in the primary ECM. N>25. Error bars=SEM.

FIG. 21 shows immunofluorescent staining of pMLC (green), F-actin (phalloidin, red), and DAPI (blue) in top-panel and paxillin (red) and DAPI (blue) in bottom-panel for MCF10A cell monolayers on the secondary ECM after 2 days of migration (post-priming). Repeated from FIG. 20 with higher resolution to better visualize actin fibers and punctate focal adhesions. Scale bar=50 μm.

FIG. 22A-FIG. 22D shows the number of cells after proliferation inhibition. (FIG. 22A-FIG. 22D) Normalized number of cells within a defined region of interest in primary and secondary ECMs at time t=16 h and t=32 h from t=0, the start of migration (post-priming), for thymidine-treated and untreated cells. Normalization performed relative to the number of cells in the Rol at t=0. Horizontal brackets denote statistical significance (P<0.05). Error bars=SEM.

FIG. 23A-FIG. 23G shows memory-dependent migration is not regulated by proliferation or long-distance signal transmission. Representative leading-edge tracks of monolayers of MCF10A cells after treatment with (FIG. 23A) 2 mM thymidine, a proliferation inhibitor, and (FIG. 23D) 4 mM EGTA, a calcium chelator, recorded for 12 h (3 h interval) in the secondary ECM, with color-coding for migration speed. Arrows indicate direction of migration. (FIG. 23B) Average leading edge migration speed for proliferation-inhibited cells. Columns with dashed outline represent the migration speed for control untreated MCF10A cells. N>15. (FIG. 23C) Average spreading area of individual cells in the secondary ECM with and without proliferation inhibition. N>30. (FIG. 23E) Average leading edge migration speed for EGTA-treated cells. N>15. (FIG. 23F) Immunofluorescent staining for E-cadherin (green) and DAPI (blue) in untreated and EGTA-treated MCF10A cells showing dysfunctional cell-cell junctions after EGTA treatment. Scale bars=100 mm. (FIG. 23G) After 3-day priming and additional 1 day of migration in the secondary ECM, the primary ECM region is entirely removed. Leading-edge migration speed in the secondary ECM (right panel) shows preservation of memory-dependent migration despite a complete loss of communication with the primary region. N>15. Horizontal brackets denote statistical significance, p<0.05. Error bars=SEM.

FIG. 24A-FIG. 24C shows YAP activity depends on past ECM stiffness. (FIG. 24A) Immunofluorescent staining of MCF10A cells for YAP (green) and DAPI (blue) illustrating the subcellular localization of YAP for the monolayer migrating on secondary ECM, after priming. Scale bar=50 mm. (FIG. 24B, FIG. 24C) Average nuclear-to-cytoplasmic ratio of the YAP fluorescent intensity for MCF10A, MCF7, and A431 cells within the monolayer. *p<0.05 with respect to control ECMs of homogeneous stiffness. Error bars=SEM. N>40.

FIG. 25A-FIG. 25B shows representative images of YAP expression in MCF7 and A431 cells. Immunofluorescent staining of MCF-7 (FIG. 25A) and A431 (FIG. 25B) cells for YAP (green) and DAPI (blue), illustrates the subcellular localization of YAP for the monolayer migrating on soft and stiff secondary ECM, after priming on soft or stiff ECMs. Scale bar=50 μm.

FIG. 26A-FIG. 26H shows memory-independent collective migration of YAP-depleted MCF10A cells. (FIG. 26A) Leading edge tracks of monolayer of YAP-depleted MCF10A cells are plotted at 3 h interval during their post-priming migration on secondary ECMs. Immunofluorescent staining for F-actin (red), pMLC (green), paxillin (red), and DAPI (blue). Scale bars=100 mm. (FIG. 26B) MCF10A cells expressing either shSCRM (wt) or shYAP RNAi were lysed and subjected to Western blotting with anti-YAP and anti-Actin antibodies. Average (FIG. 26C) leading edge migration speed, (FIG. 26D) correlation length, and (FIG. 26E) order parameter, N>15, and normalized (FIG. 26F) pMLC expression and (FIG. 26G) actin alignment, N>40, and (FIG. 26H) FA area, N>20, for varying ECM configurations. Columns with dashed outline represent corresponding values for wildtype MCF10A cells. Horizontal brackets denote statistical significance (p<0.05). ns=no significant difference. Error bars=SEM.

FIG. 27A-FIG. 27B shows monolayer dynamics in YAP-depleted MCF10A cells. (FIG. 27A) Heatmap showing the spatial distribution of velocity magnitude (top row), vector field describing the direction of velocity vectors (middle row), and order parameter (bottom row) at a given time instant for YAP-depleted MCF10A cell monolayer migrating on soft (left column) and stiff (right column) secondary ECMs, after priming on either stiff or soft primary ECMs. (FIG. 27B) Position-time kymographs of velocity magnitude and order parameter obtained from PIV analysis for corresponding ECM conditions described above to demonstrate the time evolution of monolayer motion. Kymographs were computed by averaging the velocity magnitude and order parameter of individual velocity vectors in the x direction over the y coordinate for every time point.

FIG. 28 shows immunofluorescent staining of pMLC (green), F-actin (phalloidin, red), and DAPI (blue) in top-panel and paxillin (red) and DAPI (blue) in bottom-panel for YAP-depleted MCF10A cell monolayers on the secondary ECM after 2 days of migration (post-priming). Repeated from FIG. 26 with higher resolution to better visualize actin fibers and punctate focal adhesions. Scale bar=50 μm.

FIG. 29A-FIG. 29B shows time progression of YAP nuclear localization on stiff ECM. (FIG. 29A) Average nuclear-to-cytoplasmic ratio of the YAP fluorescent intensity for MCF10A cells cultured on stiff ECM (50 kPa) located near the monolayer boundary (within 0.4 mm; blue line), within the monolayer (˜1 mm away from the monolayer boundary; red line), and overall average (regardless of location relative to the monolayer boundary; gray line) as a function of time after cell seeding. Mean values were obtained by analyzing >40 cells from >4 different fields of views from >2 experiments. *p<0.05 with respect to the 6 h data point. Error bars=SEM. (FIG. 29B) Immunofluorescent staining of MCF10A cells for YAP (green) and DAPI (blue) illustrating the nuclear localization of YAP for the monolayer cultured on stiff ECM, fixed at 6 h, 24 h, 48 h, and 72 h after seeding. Scale bar=100 μm.

FIG. 30A-FIG. 30B shows conceptual framework for memory regulation. (FIG. 30A) Priming-dependent YAP activity regulates cellular forces and dictates the memory-dependent migration. (FIG. 30B) YAP-depletion abrogates memory, but direct FA-mediated contact with the immediate ECM preserves mechanosensitivity.

FIG. 31 is a schematic describing the fabrication steps of 2D substrates of heterogeneous stiffness through modular polymerization of PA solutions of distinct compositions, resulting in dissimilar ECM stiffness in adjoining primary and secondary regions (see also FIG. 9, FIG. 13, FIG. 23).

FIG. 32 shows representative leading-edge tracks of monolayers of MCF10A cells recorded for 12 h (3 h interval) in the secondary ECM after 3-day priming, with color-coding for migration speed. Arrows indicate direction of migration. Scale bar=100 mm (see also FIG. 15).

FIG. 33 is a series of images of soft-primed and stiff-primed YAP-depleted breast epithelial cells (see also FIG. 26).

FIG. 34 is an illustration showing a 3D device and 3D breast tumor invasion due to mechanical memory (similarly, see e.g., FIG. 7).

FIG. 35 is a series of images showing more aggressive invasion of primary mouse breast tumor organoids (containing circulating tumor cells and cancer associated fibroblasts) and collagen deformation due to stiff priming.

FIG. 36A-FIG. 36F shows illustrations of examples of screening methods.

### DETAILED DESCRIPTION OF THE INVENTION

The disclosure may be understood by reference to the following detailed description, taken in conjunction with the drawings as described below. It is noted that, for purposes of illustrative clarity, certain elements in various drawings may not be drawn to scale.

The present disclosure is based, at least in part, on the discovery that migrating cells remember their past matrix stiffness as they move across mechanically dissimilar microenvironments. As shown herein, because unpredictable trajectories of metastasis and the heterogeneous populations of cancer cells in a given tumor have severely stymied the development of robust therapeutic strategies, a device has been developed that integrates multiple steps of metastasis, from primary tumor to secondary metastatic sites, permits controlled manipulation of biomechanical properties of varied microenvironments, and allows cellular measurements at every step. Moreover, the disclosed devices mimic key features of any given microenvironment.

The problem solved by the following disclosure, is that 95% of cancer therapy targets identified in preclinical research are rejected after trials. 2D substrates for cancer cell studies are generally hard surfaces. These 2D surfaces lead to cells overexpressing numerous genes that are also needed for fast invasion and proliferation and leads to non-specific screening. 3D matrices better mimic the in vivo microenvironment and thus the gene expression profile would mimic an environment closer to reality which would yield more reliable targets. Because cancer cells migrate from stiffened tumor to softer healthy tissues, the following disclosure provides for solutions for devices that can mimic this tumor microenvironment. Existing 3D matrices ignore this cancer trajectory. The current stiff tumor-like 3D matrices have several drawbacks. Cancer cells on current 3D matrices have been shown to overexpress numerous signals (e.g., RhoA, ROCK, YAP, SNAIL, TWIST, pMLC, MMPs), all of which can promote aggressive invasion, proliferation. Current 3D matrices provide the wrong targets. In reality, cancer cell leave the stiff tumor and invade through the softer healthy tissue. Through plasticity and quick adaptation, cancer cells won't express many of these mechano-sensitive targets in the soft tissue. The drugs developed in tumor-mimicking stiff 3D matrices are likely to be ineffective for cancer cells that moved to the soft tissues. Over 90% cancer deaths are due to metastasis, not the primary tumor. As such, this disclosure provides for the generation of cancer cells that mimic this metastatic cancer cell environment. Because the signals that stay activated after the cells move to healthy/soft matrix are the ones that should be focused on for therapeutic screening of cancer metastases, this is not possible to in any existing 2D or 3D systems.

The present disclosure solves this problem with methods and devices that capture both the past and present matrices (gradient stiffness in 3D) to narrow the targets for drug screening. By allowing the cells to retain the mechanical memory of past stiff tumor matrix, it is possible to extract these cells and only choose the signals that are still active. This novel approach narrows the list to persistently aggressive signals.

Out of hundreds of stiffness-sensitive signaling pathways, some of these possess a mechanical memory of past stiffness. Cancer cells use those pathways to exploit past stiffness and persistently invade through the soft matrix. The disclosed methods and device improves in vitro drug testing by allowing continuous tracking of drug effects on cancer cells as the move through dissimilar environments along the invasion trajectory.

Provided herein are devices to improve in vitro drug testing by allowing continuous tracking of drug effects on cancer cells as they move through dissimilar environments along the cancer invasion trajectory, from primary tumor to secondary metastatic sites, within one system. Throughout the multi-step metastatic trajectory, cancer cells continually encounter new 3D microenvironments defined by numerous biomechanical properties beyond just stiffness, such as dimensionality, protein composition, fiber microstructure, and native cell types.

Beyond the immediate 3D environment surrounding cells, the cell state in any given step of metastasis may depend on cell states in previous time points. This preconditioning of cells by their primary ECM may be referred to as the ‘mechanical memory’ of migratory cells. While reductionist experimental setups with tunable ECM properties allow direct interrogation of specific bio-chemo-mechanical perturbations, these approaches, by design, ignore the complexity and heterogeneity of in vivo situations. On the other hand, experimental setups that most closely mimic in vivo situations are too heterogeneous, such that it becomes hard to decouple specific influences of individual environmental parameters. The invasion trajectory devices provided herein provide a balance between the reductionist and the highly heterogeneous approaches.

Plasticity in motile cells is manifested by variable modes of migration, e.g., mesenchymal, amoeboid, and collective, depending on the surrounding microenvironment. In particular, cancer cells are uniquely equipped to exploit their plasticity to successfully adapt to foreign microenvironments and drive the relentless tumor invasion through distinct tissues. Over the course of the multi-step tumor invasion trajectory, dynamic cell-ECM interactions and adaptation to mechanically diverse microenvironments lead to unpredictable routes of cancer metastasis and heterogeneity in secondary tumors, both of which have severely stymied the development of robust therapeutic strategies. Without being limited to a particular theory, the mechanics-regulated state of cells may persist even after they migrate into a new environment. The mechanical properties of the tumor microenvironment may mechanically “train” the escaping cells, impacting their future ability to metastasize. Therefore, a drug testing platform should take into account the diverse microenvironment and cellular plasticity/memory of cancer cells when testing the efficacy of new cancer drugs.

Provided herein is an in vitro platform that integrates multiple steps of metastasis, from primary tumor to secondary metastatic sites, permits controlled manipulation of biomechanical properties of varied microenvironments, allows cellular measurements at every step, and allows continuous tracking of cancer cells throughout the invasion process. Moreover, these devices mimic key features of any given microenvironment, e.g., undergoing EMT on a primary tumor matrix, migration through stromal tissue, circulation, and potential invasion into a secondary metastatic location. The device may mimic these regions in terms of cell types, extra-cellular matrices (ECMs), and related mechanical parameters. In an aspect, the devices herein may apply to all cancer types. In one aspect, they may be designed around the problem of breast cancer metastases to different secondary locations.

Within this device, cells in the secondary sites are pre-conditioned by prior culture in primary tumor-like environments, recapitulating the in vivo journey of cancer cells. Hence, cellular measurements and drug analyses conducted in this system are likely to be more representative of in vivo situations than standard in vitro systems in which cells simply go from culture to substrate. In addition, reliability of drug treatments at the primary tumor site can be better verified in this system, by tracking how drug effects persist through the invasion trajectory over time and influence tumor growth at the secondary site.

The invasion trajectory device may be a contiguous substrate with distinct regions. The invasion trajectory device may include at least two distinct regions. In various aspects, the invasion trajectory device may include two regions (2D), three regions (3D), four regions (4D), or any number of regions needed to mimic the microenvironment of a cell invasion trajectory. In an aspect, each region of the invasion trajectory may mimic a microenvironment in the invasion trajectory. Non-limiting examples of microenvironments that may be represented by the regions of the invasion trajectory device include a primary tumor site, stromal tissue, vasculature, a secondary metastatic site, and any location in which a cell might migrate to from a primary site. In one aspect, for example, the primary tumor site may be a breast tumor and the secondary metastatic site may be lung tissue, brain tissue, spinal tissue, or any other tissue in which a breast tumor may metastasize to.

In an aspect, a region of the invasion trajectory device may mimic the microenvironment of the extracellular matrix (ECM) at a given location in the body. The microenvironment may be characterized by cell types (i.e., macrophages, fibroblasts, and cancer stem cells), proteins, extracellular molecules, and related mechanical parameters, such as stiffness, porosity, geometry, dimensionality, and fibrosity. In one aspect, the invasion trajectory device may have heterogeneous stiffness such that each region of the device has a different stiffness.

The invasion trajectory device may have a rectangular shape, a circular shape, or any shape which may be compartmentalized with more than one region. For a device having a rectangular shape, the regions may be rectangular in shape and may be linearly adjacent to one another. For a device having a circular shape, the regions may be concentric, such that the primary region may surrounded by the secondary region or any subsequent region.

The invasion trajectory device may have a height ranging from about 50 μm to about 200 μm. In various aspects, the height may range from about 50 μm to about 100 μm, from about 75 μm to about 125 μm, from about 100 μm to about 150 μm, from about 125 μm to about 175 μm, or from about 150 μm to about 200 μm. In various aspects, the height can be about 50 μm; about 51 μm; about 52 μm; about 53 μm; about 54 μm; about 55 μm; about 56 μm; about 57 μm; about 58 μm; about 59 μm; about 60 μm; about 61 μm; about 62 μm; about 63 μm; about 64 μm; about 65 μm; about 66 μm; about 67 μm; about 68 μm; about 69 μm; about 70 μm; about 71 μm; about 72 μm; about 73 μm; about 74 μm; about 75 μm; about 76 μm; about 77 μm; about 78 μm; about 79 μm; about 80 μm; about 81 μm; about 82 μm; about 83 μm; about 84 μm; about 85 μm; about 86 μm; about 87 μm; about 88 μm; about 89 μm; about 90 μm; about 91 μm; about 92 μm; about 93 μm; about 94 μm; about 95 μm; about 96 μm; about 97 μm; about 98 μm; about 99 μm; about 100 μm; about 101 μm; about 102 μm; about 103 μm; about 104 μm; about 105 μm; about 106 μm; about 107 μm; about 108 μm; about 109 μm; about 110 μm; about 111 μm; about 112 μm; about 113 μm; about 114 μm; about 115 μm; about 116 μm; about 117 μm; about 118 μm; about 119 μm; about 120 μm; about 121 μm; about 122 μm; about 123 μm; about 124 μm; about 125 μm; about 126 μm; about 127 μm; about 128 μm; about 129 μm; about 130 μm; about 131 μm; about 132 μm; about 133 μm; about 134 μm; about 135 μm; about 136 μm; about 137 μm; about 138 μm; about 139 μm; about 140 μm; about 141 μm; about 142 μm; about 143 μm; about 144 μm; about 145 μm; about 146 μm; about 147 μm; about 148 μm; about 149 μm; about 150 μm; about 151 μm; about 152 μm; about 153 μm; about 154 μm; about 155 μm; about 156 μm; about 157 μm; about 158 μm; about 159 μm; about 160 μm; about 161 μm; about 162 μm; about 163 μm; about 164 μm; about 165 μm; about 166 μm; about 167 μm; about 168 μm; about 169 μm; about 170 μm; about 171 μm; about 172 μm; about 173 μm; about 174 μm; about 175 μm; about 176 μm; about 177 μm; about 178 μm; about 179 μm; about 180 μm; about 181 μm; about 182 μm; about 183 μm; about 184 μm; about 185 μm; about 186 μm; about 187 μm; about 188 μm; about 189 μm; about 190 μm; about 191 μm; about 192 μm; about 193 μm; about 194 μm; about 195 μm; about 196 μm; about 197 μm; about 198 μm; about 199 μm; or about 200 μm. Recitation of each of these discrete values is understood to include ranges between each value. Recitation of each range is understood to include discrete values within the range.

Each region in the device may be between about 2 mm and about 7 mm. In various aspects, the length and/or width of each region may range from about 2 mm to about 4 mm, from about 3 mm to about 5 mm, from about 4 mm to about 6 mm, or from about 5 mm to about 7 mm. In various aspects, the length and/or width of each region can be about 0.1 mm; about 0.2 mm; about 0.3 mm; about 0.4 mm; about 0.5 mm; about 0.6 mm; about 0.7 mm; about 0.8 mm; about 0.9 mm; about 1 mm; about 1.1 mm; about 1.2 mm; about 1.3 mm; about 1.4 mm; about 1.5 mm; about 1.6 mm; about 1.7 mm; about 1.8 mm; about 1.9 mm; about 2 mm; about 2.1 mm; about 2.2 mm; about 2.3 mm; about 2.4 mm; about 2.5 mm; about 2.6 mm; about 2.7 mm; about 2.8 mm; about 2.9 mm; about 3 mm; about 3.1 mm; about 3.2 mm; about 3.3 mm; about 3.4 mm; about 3.5 mm; about 3.6 mm; about 3.7 mm; about 3.8 mm; about 3.9 mm; about 4 mm; about 4.1 mm; about 4.2 mm; about 4.3 mm; about 4.4 mm; about 4.5 mm; about 4.6 mm; about 4.7 mm; about 4.8 mm; about 4.9 mm; about 5 mm; about 5.1 mm; about 5.2 mm; about 5.3 mm; about 5.4 mm; about 5.5 mm; about 5.6 mm; about 5.7 mm, about 5.8 mm, about 5.9 mm, about 6 mm, about 6.1 mm; about 6.2 mm; about 6.3 mm; about 6.4 mm; about 6.5 mm; about 6.6 mm; about 6.7 mm; about 6.8 mm; about 6.9 mm; about 7 mm; about 7.1 mm; about 7.2 mm; about 7.3 mm; about 7.4 mm; about 7.5 mm; about 7.6 mm; about 7.7 mm; about 7.8 mm; about 7.9 mm; about 8 mm; about 8.1 mm; about 8.2 mm; about 8.3 mm; about 8.4 mm; about 8.5 mm; about 8.6 mm; about 8.7 mm; about 8.8 mm; about 8.9 mm; about 9 mm; about 9.1 mm; about 9.2 mm; about 9.3 mm; about 9.4 mm; about 9.5 mm; about 9.6 mm; about 9.7 mm; about 9.8 mm; about 9.9 mm; about 10 mm, about 10.1 mm; about 10.2 mm; about 10.3 mm; about 10.4 mm; about 10.5 mm; about 10.6 mm; about 10.7 mm; about 10.8 mm; about 10.9 mm; about 11 mm; about 11.1 mm; about 11.2 mm; about 11.3 mm; about 11.4 mm; about 11.5 mm; about 11.6 mm; about 11.7 mm; about 11.8 mm, about 11.9 mm, about 12 mm; about 12.1 mm; about 12.2 mm; about 12.3 mm; about 12.4 mm; about 12.5 mm; about 12.6 mm; about 12.7 mm; about 12.8 mm; about 12.9 mm; about 13 mm; about 13.1 mm; about 13.2 mm; about 13.3 mm; about 13.4 mm; about 13.5 mm; about 13.6 mm; about 13.7 mm; about 13.8 mm; about 13.9 mm; about 14 mm; about 14.1 mm; about 14.2 mm; about 14.3 mm; about 14.4 mm; about 14.5 mm; about 14.6 mm; about 14.7 mm; about 14.8 mm; about 14.9 mm; about 15 mm; about 15.1 mm; about 15.2 mm; about 15.3 mm; about 15.4 mm; about 15.5 mm; about 15.6 mm; about 15.7 mm; about 15.8 mm; about 15.9 mm; about 16 mm; about 16.1 mm; about 16.2 mm; about 16.3 mm; about 16.4 mm; about 16.5 mm; about 16.6 mm; about 16.7 mm; about 16.8 mm; about 16.9 mm; about 17 mm; about 17.1 mm; about 17.2 mm; about 17.3 mm; about 17.4 mm; about 17.5 mm; about 17.6 mm; about 17.7 mm; about 17.8 mm; about 17.9 mm; about 18 mm; about 18.1 mm; about 18.2 mm; about 18.3 mm; about 18.4 mm; about 18.5 mm; about 18.6 mm; about 18.7 mm; about 18.8 mm; about 18.9 mm; about 19 mm; about 19.1 mm; about 19.2 mm; about 19.3 mm; about 19.4 mm; about 19.5 mm; about 19.6 mm; about 19.7 mm; about 19.8 mm; about 19.9 mm; or about 20 mm. Recitation of each of these discrete values is understood to include ranges between each value. Recitation of each range is understood to include discrete values within the range.

Total length or width of the device may be between about 12 mm and about 50 mm. In various aspects, the length or width of the device may range from about 12 mm to about 17 mm, from about 15 mm to about 25 mm, from about 20 mm to about 30 mm, from about 25 mm to about 35 mm, from about 30 mm to about 40 mm, from about 35 mm to about 45 mm, or from about 40 mm to about 50 mm. In various aspects, the length or width of the device can be about 1 mm; about 1.5 mm; about 2 mm; about 2.5 mm; about 3 mm, about 3.5 mm, about 4 mm; about 4.5 mm; about 5 mm; about 5.5 mm; about 6 mm; about 6.5 mm; about 7 mm; about 7.5 mm; about 8 mm; about 8.5 mm; about 9 mm; about 9.5 mm; about 10 mm; about 10.5 mm, about 11 mm; about 11.5 mm, about 12 mm; about 12.5 mm; about 13 mm, about 13.5 mm; about 14 mm; about 14.5 mm; about 15 mm; about 15.5 mm; about 16 mm; about 16.5 mm; about 17 mm; about 17.5 mm; about 18 mm; about 18.5 mm; about 19 mm; about 19.5 mm; about 20 mm; about 20.5 mm; about 21 mm; about 21.5 mm; about 22 mm; about 22.5 mm; about 23 mm; about 23.5 mm; about 24 mm; about 24.5 mm; about 25 mm; about 25.5 mm; about 26 mm; about 26.5 mm; about 27 mm; about 27.5 mm; about 28 mm; about 28.5 mm; about 29 mm; about 29.5 mm; about 30 mm; about 30.5 mm; about 31 mm; about 31.5 mm; about 32 mm; about 32.5 mm; about 33 mm; about 33.5 mm; about 34 mm; about 34.5 mm; about 35 mm; about 35.5 mm; about 36 mm; about 36.5 mm; about 37 mm; about 37.5 mm; about 38 mm; about 38.5 mm; about 39 mm; about 39.5 mm; about 40 mm; about 40.5 mm; about 41 mm; about 41.5 mm; about 42 mm; about 42.5 mm; about 43 mm; about 43.5 mm; about 44 mm; about 44.5 mm; about 45 mm; about 45.5 mm; about 46 mm; about 46.5 mm; about 47 mm; about 47.5 mm; about 48 mm; about 48.5 mm; about 49 mm; about 49.5 mm; or about 50 mm. Recitation of each of these discrete values is understood to include ranges between each value. Recitation of each range is understood to include discrete values within the range.

The invasion trajectory device may further include at least one microchannel through at least one region of the device. In an aspect, the microchannels may pass through more than one region of the device. In another aspect, the microchannels may pass through all regions of the device. In yet another aspect, the microchannels may be located in a region between the primary region and the secondary region. The microchannels may extend parallel to the length of the device, perpendicular to the length of the device, radially outward from the center of the device, or in any direction through the device. The migrating cells may be confined by the microchannels, which may limit the direction of movement of the cells. The microchannels may be used to mimic 3-dimenensional constraints on the cells in the body. In an aspect, the secondary region may be a 3D ECM created by polymerizing a thicket secondary or other region around or adjacent to a primary region. The 3D ECM may be degradable, such as comprising collagen or fibrin.

The migrating cells may also migrate through microchannels. For example, microchannels may be used to mimic circulation through the vasculature between the primary side and the secondary site. In an aspect, the cells may also migrate through a region representing stromal tissue before entering the microchannels. The microchannels may aid in measuring a cancer cell's plasticity and memory for varying travel distances from primary to secondary ECMs.

The microchannels, or flow channels, may have varying shapes and sizes. The shape of the microchannels may generally be linear, curved, serpentine, tortuous, irregularly shaped, or any configuration that may represent blood vessels. In an aspect, the curvature in channel geometry attempts to mimic the tortuous circulation of cancer cells. The device may include at least one channel, at least two channels, at least three channels, or any number of channels needed to represent metastasis. Each channel in the device may have a different length, width, height, or shape than the other channels in the device. Each channel in the device may have an average length, width, or height according to the below microchannel values.

The microchannels may range in width from about 100 μm to about 500 μm. In various aspects, the channel width may range from about 10 μm to about 40 μm, from about 30 μm to about 50 μm, from about 40 μm to about 60 μm, from about 50 μm to about 70 μm, from about 60 μm to about 80 μm, from about 70 μm to about 90 μm, from about 80 μm to about 100 μm, from about 90 μm to about 110 μm, from about 100 μm to about 150 μm, from about 125 μm to about 175 μm, from about 150 μm to about 200 μm, from about 175 μm to about 225 μm, from about 200 μm to about 300 μm, from about 250 μm to about 350 μm, from about 300 μm to about 400 μm, from about 350 μm to about 450 μm, or from about 400 μm to about 500 μm. In various aspects, the channel width can be about 1 μM; about 10 μM; about 20 μM; about 30 μM; about 40 μM; about 50 μM; about 60 μM; about 70 μM; about 80 μM; about 90 μM; about 100 μM; about 110 μM; about 120 μM; about 130 μM; about 140 μM; about 150 μM; about 160 μM; about 170 μM; about 180 μM; about 190 μM; about 200 μM; about 210 μM; about 220 μM; about 230 μM; about 240 μM; about 250 μM; about 260 μM; about 270 μM; about 280 μM; about 290 μM; about 300 μM; about 310 μM; about 320 μM; about 330 μM; about 340 μM; about 350 μM; about 360 μM; about 370 μM; about 380 μM; about 390 μM; about 400 μM; about 410 μM; about 420 μM; about 430 μM; about 440 μM; about 450 μM; about 460 μM; about 470 μM; about 480 μM; about 490 μM; or about 500 μM. Recitation of each of these discrete values is understood to include ranges between each value. Recitation of each range is understood to include discrete values within the range.

The length of the channels may range from about 2 mm to about 50 mm. In various aspects, the length of the channels may range from about 2 mm to about 5 mm, from about 3 mm to about 6 mm, from about 5 mm to about 10 mm, from about 7 mm to about 12, mm, from about 10 mm to about 20 mm, from about 15 mm to about 25 mm, from about 20 mm to about 30 mm, from about 25 mm to about 35 mm, from about 30 mm to about 40 mm, from about 35 mm to about 45 mm, or from about 40 mm to about 50 mm. In various aspects, the length of the channels can be about 1 mm; about 2 mm; about 3 mm; about 4 mm; about 5 mm; about 6 mm; about 7 mm; about 8 mm; about 9 mm; about 10 mm; about 11 mm; about 12 mm; about 13 mm; about 14 mm; about 15 mm; about 16 mm, about 17 mm; about 18 mm; about 19 mm, about 20 mm; about 21 mm; about 22 mm; about 23 mm, about 24 mm; about 25 mm, about 26 mm, about 27 mm; about 28 mm; about 29 mm; about 30 mm; about 31 mm; about 32 mm, about 33 mm; about 34 mm, about 35 mm; about 36 mm, about 37 mm, about 38 mm; about 39 mm; about 40 mm; about 41 mm; about 42 mm; about 43 mm, about 44 mm; about 45 mm; about 46 mm; about 47 mm; about 48 mm; about 49 mm; or about 50 mm. Recitation of each of these discrete values is understood to include ranges between each value. Recitation of each range is understood to include discrete values within the range.

The height of the channel may range from about 20 μm to about 100 μm, from about 30 μm to about 50 μm, from about 40 μm to about 60 μm, from about 50 μm to about 70 μm, from about 60 μm to about 80 μm, from about 70 μm to about 90 μm, or from about 80 μm to about 100 μm. In various aspects, the height of the channel can be about 10 μm; about 11 μm; about 12 μm; about 13 μm; about 14 μm; about 15 μm; about 16 μm; about 17 μm; about 18 μm; about 19 μm; about 20 μm; about 21 μm; about 22 μm; about 23 μm; about 24 μm; about 25 μm; about 26 μm; about 27 μm; about 28 μm; about 29 μm; about 30 μm; about 31 μm; about 32 μm; about 33 μm; about 34 μm; about 35 μm; about 36 μm; about 37 μm; about 38 μm; about 39 μm; about 40 μm; about 41 μm; about 42 μm; about 43 μm; about 44 μm; about 45 μm; about 46 μm; about 47 μm; about 48 μm; about 49 μm; about 50 μm; about 51 μm; about 52 μm; about 53 μm; about 54 μm; about 55 μm; about 56 μm; about 57 μm; about 58 μm; about 59 μm; about 60 μm; about 61 μm; about 62 μm; about 63 μm; about 64 μm; about 65 μm; about 66 μm; about 67 μm; about 68 μm; about 69 μm; about 70 μm; about 71 μm; about 72 μm; about 73 μm; about 74 μm; about 75 μm; about 76 μm; about 77 μm; about 78 μm; about 79 μm; about 80 μm; about 81 μm; about 82 μm; about 83 μm; about 84 μm; about 85 μm; about 86 μm; about 87 μm; about 88 μm; about 89 μm; about 90 μm; about 91 μm; about 92 μm; about 93 μm; about 94 μm; about 95 μm; about 96 μm; about 97 μm; about 98 μm; about 99 μm; or about 100 μm. In general, the height of the channel may not be more than double the width of the channel. Recitation of each of these discrete values is understood to include ranges between each value. Recitation of each range is understood to include discrete values within the range.

The invasion trajectory device may be fabricated using a polymer with a tunable stiffness. In various aspects, the tunable stiffness polymer may include polyacrylamide (PA), polydimethylsiloxane (PDMS), styrene, N-Vinylpyrrolidone, acrylates, alginate, agarose, collagen, fibrin, gellan (e.g., fibrillary gellan, gellan gum), gelatin, hyaluronic acid, chitosan, methylcellulose, hyaluronan, elastin, laminin, fibronectin, other naturally derived polymers, a semi-flexible polyelectrolyte, a copolymer, such as poly(methacrylamide-co-methacrylate), poly(vinyl alcohol) (PVA), polyacrylamide (PA), polydimethylsiloxane (PDMS), poly(ethylene glycol) (PEG), poly(lactic-co-glycolic acid) (PLGA)), sucrose acrylate, N-Vinylpyrrolidone, N-vinyl-2-pyrrolidinone, polymers including monomers of bisacrylamide, acrylate, acrylamide, styrene, vinyl, acrylic acid, salts of acrylic acid such as sodium and potassium acrylates or sodium and sulfopropyl acrylates, or 2-hydroxyethyl methacrylate or any polymer where the stiffness may be manipulated.

As another example, In an aspect, photopolymerization may be used to generate the variation in stiffness. For example, a photopolimerizable polymer solution may be mixed with a photoinitiator and be polymerized through a mask under UV exposure, resulting in a substrate with regions of dissimilar stiffness. In another aspect, the regions of heterogeneous stiffness may be generated by controlled mixing of polymer ratios. Microfluidic mixing may be used to combine different ratios of a monomer and a crosslinker such that when the mixed solution is polymerized, the varied ratios will result in distinct regions stiffness on a continuous substrate. There may be a step-wise difference in stiffness between neighboring regions, there may be a gradient of stiffness across regions, or regions of varying stiffness may be separated by a distance. In an aspect, the regions of varying stiffness may be separated by a region with microchannels, representing the vasculature.

The stiffness of the various regions of the invasion trajectory device may range from about 0.08 kPa to about 120 kPa. In various aspects, the stiffness of the regions of the invasion trajectory device may range from about 0.08 kPa to about 1 kPa, from about 0.5 kPa to about 5 kPa, from about 1 kPa to about 10 kPa, from about 5 kPa to about 15 kPa, from about 10 kPa to about 20 kPa, from about 15 kPa to about 25 kPa, from about 20 kPa to about 40 kPa, from about 30 kPa to about 50 kPa, from about 40 kPa to about 60 kPa, from about 50 kPa to about 100 kPa, or from about 75 kPa to about 120 kPa. In other aspects, the stiffness of the regions of the invasion trajectory device may be about 0.1 kPa; about 0.2 kPa; about 0.3 kPa; about 0.4 kPa; about 0.5 kPa; about 0.6 kPa; about 0.7 kPa; about 0.8 kPa; about 0.9 kPa; about 1 kPa; about 1.1 kPa; about 1.2 kPa; about 1.3 kPa; about 1.4 kPa; about 1.5 kPa; about 1.6 kPa; about 1.7 kPa; about 1.8 kPa; about 1.9 kPa; about 2 kPa; about 2.1 kPa; about 2.2 kPa; about 2.3 kPa; about 2.4 kPa; about 2.5 kPa; about 2.6 kPa; about 2.7 kPa; about 2.8 kPa; about 2.9 kPa; about 3 kPa; about 3.1 kPa; about 3.2 kPa; about 3.3 kPa; about 3.4 kPa; about 3.5 kPa; about 3.6 kPa; about 3.7 kPa; about 3.8 kPa; about 3.9 kPa; about 4 kPa; about 4.1 kPa; about 4.2 kPa; about 4.3 kPa; about 4.4 kPa; about 4.5 kPa; about 4.6 kPa; about 4.7 kPa; about 4.8 kPa; about 4.9 kPa; about 5 kPa; about 5.1 kPa; about 5.2 kPa; about 5.3 kPa; about 5.4 kPa; about 5.5 kPa; about 5.6 kPa; about 5.7 kPa; about 5.8 kPa; about 5.9 kPa; about 6 kPa; about 6.1 kPa; about 6.2 kPa; about 6.3 kPa; about 6.4 kPa; about 6.5 kPa; about 6.6 kPa; about 6.7 kPa; about 6.8 kPa; about 6.9 kPa; about 7 kPa; about 7.1 kPa; about 7.2 kPa; about 7.3 kPa; about 7.4 kPa; about 7.5 kPa; about 7.6 kPa; about 7.7 kPa; about 7.8 kPa; about 7.9 kPa; about 8 kPa; about 8.1 kPa; about 8.2 kPa; about 8.3 kPa; about 8.4 kPa; about 8.5 kPa; about 8.6 kPa; about 8.7 kPa; about 8.8 kPa; about 8.9 kPa; about 9 kPa; about 9.1 kPa; about 9.2 kPa; about 9.3 kPa; about 9.4 kPa; about 9.5 kPa; about 9.6 kPa; about 9.7 kPa; about 9.8 kPa; about 9.9 kPa; about 10 kPa; about 10.1 kPa; about 10.2 kPa; about 10.3 kPa; about 10.4 kPa; about 10.5 kPa; about 10.6 kPa; about 10.7 kPa; about 10.8 kPa; about 10.9 kPa; about 11 kPa; about 11.1 kPa; about 11.2 kPa; about 11.3 kPa; about 11.4 kPa; about 11.5 kPa; about 11.6 kPa; about 11.7 kPa; about 11.8 kPa; about 11.9 kPa; about 12 kPa; about 12.1 kPa; about 12.2 kPa; about 12.3 kPa; about 12.4 kPa; about 12.5 kPa; about 12.6 kPa; about 12.7 kPa; about 12.8 kPa; about 12.9 kPa; about 13 kPa; about 13.1 kPa; about 13.2 kPa; about 13.3 kPa; about 13.4 kPa; about 13.5 kPa; about 13.6 kPa; about 13.7 kPa; about 13.8 kPa; about 13.9 kPa; about 14 kPa; about 14.1 kPa; about 14.2 kPa; about 14.3 kPa; about 14.4 kPa; about 14.5 kPa; about 14.6 kPa; about 14.7 kPa; about 14.8 kPa; about 14.9 kPa; about 15 kPa; about 15.1 kPa; about 15.2 kPa; about 15.3 kPa; about 15.4 kPa; about 15.5 kPa; about 15.6 kPa; about 15.7 kPa; about 15.8 kPa; about 15.9 kPa; about 16 kPa; about 16.1 kPa; about 16.2 kPa; about 16.3 kPa; about 16.4 kPa; about 16.5 kPa; about 16.6 kPa; about 16.7 kPa; about 16.8 kPa; about 16.9 kPa; about 17 kPa; about 17.1 kPa; about 17.2 kPa; about 17.3 kPa; about 17.4 kPa; about 17.5 kPa; about 17.6 kPa; about 17.7 kPa; about 17.8 kPa; about 17.9 kPa; about 18 kPa; about 18.1 kPa; about 18.2 kPa; about 18.3 kPa; about 18.4 kPa; about 18.5 kPa; about 18.6 kPa; about 18.7 kPa; about 18.8 kPa; about 18.9 kPa; about 19 kPa; about 19.1 kPa; about 19.2 kPa; about 19.3 kPa; about 19.4 kPa; about 19.5 kPa; about 19.6 kPa; about 19.7 kPa; about 19.8 kPa; about 19.9 kPa; about 20 kPa; about 20.1 kPa; about 20.2 kPa; about 20.3 kPa; about 20.4 kPa; about 20.5 kPa; about 20.6 kPa; about 20.7 kPa; about 20.8 kPa; about 20.9 kPa; about 21 kPa; about 21.1 kPa; about 21.2 kPa; about 21.3 kPa; about 21.4 kPa; about 21.5 kPa; about 21.6 kPa; about 21.7 kPa; about 21.8 kPa; about 21.9 kPa; about 22 kPa; about 22.1 kPa; about 22.2 kPa; about 22.3 kPa; about 22.4 kPa; about 22.5 kPa; about 22.6 kPa; about 22.7 kPa; about 22.8 kPa; about 22.9 kPa; about 23 kPa; about 23.1 kPa; about 23.2 kPa; about 23.3 kPa; about 23.4 kPa; about 23.5 kPa; about 23.6 kPa; about 23.7 kPa; about 23.8 kPa; about 23.9 kPa; about 24 kPa; about 24.1 kPa; about 24.2 kPa; about 24.3 kPa; about 24.4 kPa; about 24.5 kPa; about 24.6 kPa; about 24.7 kPa; about 24.8 kPa; about 24.9 kPa; about 25 kPa; about 26 kPa; about 27 kPa; about 28 kPa; about 29 kPa; about 30 kPa; about 31 kPa; about 32 kPa; about 33 kPa; about 34 kPa; about 35 kPa; about 36 kPa; about 37 kPa; about 38 kPa; about 39 kPa; about 40 kPa; about 41 kPa; about 42 kPa; about 43 kPa; about 44 kPa; about 45 kPa; about 46 kPa; about 47 kPa; about 48 kPa; about 49 kPa; about 50 kPa; about 51 kPa; about 52 kPa; about 53 kPa; about 54 kPa; about 55 kPa; about 56 kPa; about 57 kPa; about 58 kPa; about 59 kPa; about 60 kPa; about 61 kPa; about 62 kPa; about 63 kPa; about 64 kPa; about 65 kPa; about 66 kPa; about 67 kPa; about 68 kPa; about 69 kPa; about 70 kPa; about 71 kPa; about 72 kPa; about 73 kPa; about 74 kPa; about 75 kPa; about 76 kPa; about 77 kPa; about 78 kPa; about 79 kPa; about 80 kPa; about 81 kPa; about 82 kPa; about 83 kPa; about 84 kPa; about 85 kPa; about 86 kPa; about 87 kPa; about 88 kPa; about 89 kPa; about 90 kPa; about 91 kPa; about 92 kPa; about 93 kPa; about 94 kPa; about 95 kPa; about 96 kPa; about 97 kPa; about 98 kPa; about 99 kPa; about 100 kPa; about 101 kPa; about 102 kPa; about 103 kPa; about 104 kPa; about 105 kPa; about 106 kPa; about 107 kPa; about 108 kPa; about 109 kPa; about 110 kPa; about 111 kPa; about 112 kPa; about 113 kPa; about 114 kPa; about 115 kPa; about 116 kPa; about 117 kPa; about 118 kPa; about 119 kPa; or about 120 kPa. Recitation of each range is understood to include discrete values within the range. Recitation of each of these discrete values is understood to include ranges between each value. In an aspect, the stiffness of the primary region may be higher than the secondary region. In another aspect, the stiffness of the secondary region may be higher than the primary region.

The invasion trajectory device may be further coated with a polymer over the distinct regions of stiffness. Examples of polymer coating layers include collagen, fibrin, or any polymer which may mimic the tissue in which the cells may migrate through. In an aspect, the device may be further covered with a glass coverslip.

Cells may be initially seeded on one region of the invasion trajectory device. The initially seeded cells may be limited from migrating to a second region of the device for a period of time. During this period of time, the cells may become preconditioned to the conditions of the region, including the stiffness or other ECM properties of the region. In an aspect, the cells may be initially physically limited from migrating to a second region by a barrier, such as a stencil over the second region. In another aspect, the cells may be initially limited by the number of cells, the location of the cells on the first region, or the size of the first region, such that the time needed for the cells to migrate through the first region and to the second region would be sufficient time for the cells to become preconditioned to the first region. The cells in first region may be preconditioned for about 12 hours to about 7 days. Non-limiting examples of preconditioning times are about 12 hours, about 1 day, about 2 days, about 3 days, about 4 days, about 5 days, about 6 days, and about 7 days. Recitation of each range is understood to include discrete values within the range. Recitation of each of these discrete values is understood to include ranges between each value.

The cells can be measured (e.g., for migration, speed, correlation length, velocity vectors) after the cells are preconditioned or primed. The cells can be evaluated after the cells enter a second region (e.g., a secondary ECM). For example, the cells can be measured at about 1 hour, about 2 hours, about 3 hours, about 4 hours, about 5 hours, about 6 hours, about 7 hours, about 8 hours, about 9 hours, about 10 hours, about 11 hours, about 12 hours, about 48 hours, about 72 hours, or about 96 hours after preconditioning. As another example, cells can be allowed to enter a second region about 1 h; about 2 h; about 3 h; about 4 h; about 5 h; about 6 h; about 7 h; about 8 h; about 9 h; about 10 h; about 11 h; about 12 h; about 13 h; about 14 h; about 15 h; about 16 h; about 17 h; about 18 h; about 19 h; about 20 h; about 21 h; about 22 h; about 23 h; about 24 h; about 25 h; about 26 h; about 27 h; about 28 h; about 29 h; about 30 h; about 31 h; about 32 h; about 33 h; about 34 h; about 35 h; about 36 h; about 37 h; about 38 h; about 39 h; about 40 h; about 41 h; about 42 h; about 43 h; about 44 h; about 45 h; about 46 h; about 47 h; about 48 h; about 49 h; about 50 h; about 51 h; about 52 h; about 53 h; about 54 h; about 55 h; about 56 h; about 57 h; about 58 h; about 59 h; about 60 h; about 61 h; about 62 h; about 63 h; about 64 h; about 65 h; about 66 h; about 67 h; about 68 h; about 69 h; about 70 h; about 71 h; about 72 h; about 73 h; about 74 h; about 75 h; about 76 h; about 77 h; about 78 h; about 79 h; about 80 h; about 81 h; about 82 h; about 83 h; about 84 h; about 85 h; about 86 h; about 87 h; about 88 h; about 89 h; about 90 h; about 91 h; about 92 h; about 93 h; about 94 h; about 95 h; about 96 h; about 97 h; about 98 h; about 99 h; about 100 h; about 101 h; about 102 h; about 103 h; about 104 h; about 105 h; about 106 h; about 107 h; about 108 h; about 109 h; about 110 h; about 111 h; about 112 h; about 113 h; about 114 h; about 115 h; about 116 h; about 117 h; about 118 h; about 119 h; about 120 h; about 121 h; about 122 h; about 123 h; about 124 h; about 125 h; about 126 h; about 127 h; about 128 h; about 129 h; about 130 h; about 131 h; about 132 h; about 133 h; about 134 h; about 135 h; about 136 h; about 137 h; about 138 h; about 139 h; about 140 h; about 141 h; about 142 h; about 143 h; about 144 h; about 145 h; about 146 h; about 147 h; about 148 h; about 149 h; about 150 h; about 151 h; about 152 h; about 153 h; about 154 h; about 155 h; about 156 h; about 157 h; about 158 h; about 159 h; about 160 h; about 161 h; about 162 h; about 163 h; about 164 h; about 165 h; about 166 h; about 167 h; about 168 h; about 169 h; about 170 h; about 171 h; about 172 h; about 173 h; about 174 h; about 175 h; about 176 h; about 177 h; about 178 h; about 179 h; about 180 h; about 181 h; about 182 h; about 183 h; about 184 h; about 185 h; about 186 h; about 187 h; about 188 h; about 189 h; about 190 h; about 191 h; about 192 h; about 193 h; about 194 h; about 195 h; about 196 h; about 197 h; about 198 h; about 199 h; or about 200 h after preconditioning of the cells. Recitation of each of these discrete values is understood to include ranges between each value.

The cells seeded on the device may be cancer cells or any type of cell that is desired to be observed. In an aspect, the cells may include breast cancer cells, ovarian cancer cells, bone cancer cells, liver cancer cells, colorectal cancer cells, pancreatic cancer cells, prostate cancer cells, adrenal gland cancer cells, kidney cancer cells, lung cancer cells, skin melanoma cells, squamous carcinoma cells, brain cancer cells, T cells, dendritic cells, or any cells that may be involved with migration or metastasis. The cells may be various mammary cells, including but not limited to MCF7, MDA-MB231, and Eph4Ras. In other aspects the cells may be mammary epithelial cells, such as MCF10A cells or MCF10A variants of specific oncogenic lesions, such as MCF10DCIS, MCF10AT or with overexpressed H-Ras, ErbB2, or 14-3-3ζ or with Rho activated cells. In other aspects, the cells can be squamous carcinoma (e.g., A431). In another aspect, the cancer cells can be from a biopsy sample.

The cells may be observed for migration through the device. In an aspect, the device may be imaged at various time points to observe the migration pattern. The distance or speed that the cells or clusters of cells have migrated through the device may be measured. Molecular expressions of the migrating cells may also be measured and/or examined. Metastasis may be quantified in terms of cancer cell growth at the secondary site.

In an aspect, a 2D device may be fabricated with primary and secondary ECM regions of dissimilar stiffness on a single substrate. A colony of cancer cells may be seeded in the primary region and cellular measurements may be performed in the secondary region. In an aspect, the ECM material may be photopolymerized. A photomask may be used to create the difference in polymerization between the two ECMs such that the two ECMs have different stiffnesses. In an aspect, cells may be initially seeded on a primary EMC region and initially limited to only the primary ECM region. In this aspect, the cells may be physically limited to the primary ECM region by a polymer mold or stencil over the secondary ECM region. Without being limited to a particular theory, initially limiting the cells to the primary ECM region may provide for the cells to be preconditioned in the primary ECM before having the ability to migrate to the secondary ECM.

Various mechanical properties that define the 3D ECM are known to influence cell behavior differently. For example, collagen fibers provide contact guidance for moving cells, but high density of fibers both increases ECM stiffness and restricts cell movement due to steric hindrance. Moreover, the interaction of cancer cells with their 3D environments varies in different contexts—collectively migrating cell sheets generally stay on 2D surfaces; tubes and streams of cell clusters move through tunnel-like ECM spaces without intimate interaction with ECM fibers; single cells, smaller cell clusters, and leader cells physically remodel and degrade the fibrous ECMs. Thus, any one type of “3D” in vitro matrix platform will not permit a thorough investigation of many of these cell-ECM interactions. The invasion trajectory device introduces complexity in 3D matrices in a step-by-step manner, depending on the studied cellular phenomenon.

In various aspects, the invasion trajectory device may be used for testing various drugs. The stiffness of the first region may be comparable to the stiffness of the primary tissue site in which the cells are typically found. The stiffness of the second region may be comparable to the stiffness of the secondary tissue site in which the cells might metastasize. Before the cells migrate to the second region of the device, they may first be preconditioned in the first region with a stiffness different from the second region.

In an aspect, the migration properties of the cells may be observed both before and after the application of a drug to the cells. The observed cells' migration properties are selected from the group consisting of migration speed, migration distance, molecular expressions, and combinations thereof. A drug's effect on the cells' cellular memory or ability to metastasize may be readily observed through the device. Different drugs may be compared for their effect on the cells within the device.

Screening

Also provided are methods for screening drugs for use as cancer treatments. The present disclosure provides for a device and methods for preparing cancer cells in an environment that more closely mimics a real tumor environment.

The device and methods provided herein can be used in the screening of a variety of different candidate molecules (e.g., potentially therapeutic candidate molecules). Candidate substances for screening according to the methods described herein include, but are not limited to, chemotherapy, radiation, immunotherapy, targeted therapy, hormone therapy, fractions of tissues or cells, nucleic acids, polypeptides, siRNAs, antisense molecules, aptamers, ribozymes, triple helix compounds, antibodies, and small (e.g., less than about 2000 mw, or less than about 1000 mw, or less than about 800 mw) organic molecules or inorganic molecules including but not limited to salts or metals.

Candidate molecules encompass numerous chemical classes, for example, organic molecules, such as small organic compounds having a molecular weight of more than 50 and less than about 2,500 Daltons. Candidate molecules can comprise functional groups necessary for structural interaction with proteins, particularly hydrogen bonding, and typically include at least an amine, carbonyl, hydroxyl or carboxyl group, and usually at least two of the functional chemical groups. The candidate molecules can comprise cyclical carbon or heterocyclic structures and/or aromatic or polyaromatic structures substituted with one or more of the above functional groups.

A candidate molecule can be a compound in a library database of compounds. One of skill in the art will be generally familiar with, for example, numerous databases for commercially available compounds for screening (see e.g., ZINC database, UCSF, with 2.7 million compounds over 12 distinct subsets of molecules; Irwin and Shoichet (2005) J Chem Inf Model 45, 177-182). One of skill in the art will also be familiar with a variety of search engines to identify commercial sources or desirable compounds and classes of compounds for further testing (see e.g., ZINC database; eMolecules.com; and electronic libraries of commercial compounds provided by vendors, for example: ChemBridge, Princeton BioMolecular, Ambinter SARL, Enamine, ASDI, Life Chemicals etc.).

Candidate molecules for screening according to the methods described herein include both lead-like compounds and drug-like compounds. A lead-like compound is generally understood to have a relatively smaller scaffold-like structure (e.g., molecular weight of about 150 to about 350 kD) with relatively fewer features (e.g., less than about 3 hydrogen donors and/or less than about 6 hydrogen acceptors; hydrophobicity character xlogP of about −2 to about 4) (see e.g., Angewante (1999) Chemie Int. ed. Engl. 24, 3943-3948). In contrast, a drug-like compound is generally understood to have a relatively larger scaffold (e.g., molecular weight of about 150 to about 500 kD) with relatively more numerous features (e.g., less than about 10 hydrogen acceptors and/or less than about 8 rotatable bonds; hydrophobicity character xlogP of less than about 5) (see e.g., Lipinski (2000) J. Pharm. Tox. Methods 44, 235-249). Initial screening can be performed with lead-like compounds.

When designing a lead from spatial orientation data, it can be useful to understand that certain molecular structures are characterized as being “drug-like”. Such characterization can be based on a set of empirically recognized qualities derived by comparing similarities across the breadth of known drugs within the pharmacopoeia. While it is not required for drugs to meet all, or even any, of these characterizations, it is far more likely for a drug candidate to meet with clinical successful if it is drug-like.

Several of these “drug-like” characteristics have been summarized into the four rules of Lipinski (generally known as the “rules of fives” because of the prevalence of the number 5 among them). While these rules generally relate to oral absorption and are used to predict bioavailability of compound during lead optimization, they can serve as effective guidelines for constructing a lead molecule during rational drug design efforts such as may be accomplished by using the methods of the present disclosure.

The four “rules of five” state that a candidate drug-like compound should have at least three of the following characteristics: (i) a weight less than 500 Daltons; (ii) a log of P less than 5; (iii) no more than 5 hydrogen bond donors (expressed as the sum of OH and NH groups); and (iv) no more than 10 hydrogen bond acceptors (the sum of N and 0 atoms). Also, drug-like molecules typically have a span (breadth) of between about 8 Å to about 15 Å.

Kits

Also provided are kits. Such kits can include an agent or composition described herein and, in certain embodiments, instructions for administration. Such kits can facilitate performance of the methods described herein. When supplied as a kit, the different components of the composition can be packaged in separate containers and admixed immediately before use. Components include, but are not limited to therapeutic agents, cancer cells, a device comprising at least a first and second region having different stiffness values, optionally comprising microchannels, a polymer (e.g., a polyacrylamide), cell culture components or components that mimic biocompatible properties (e.g., collagen, fibroblasts, macrophages), or a coverglass. Such packaging of the components separately can, if desired, be presented in a pack or dispenser device which may contain one or more unit dosage forms containing the composition. The pack may, for example, comprise metal or plastic foil such as a blister pack. Such packaging of the components separately can also, in certain instances, permit long-term storage without losing activity of the components.

Kits may also include reagents in separate containers such as, for example, sterile water or saline to be added to a lyophilized active component packaged separately. For example, sealed glass ampules may contain a lyophilized component and in a separate ampule, sterile water, sterile saline or sterile each of which has been packaged under a neutral non-reacting gas, such as nitrogen. Ampules may consist of any suitable material, such as glass, organic polymers, such as polycarbonate, polystyrene, ceramic, metal or any other material typically employed to hold reagents. Other examples of suitable containers include bottles that may be fabricated from similar substances as ampules, and envelopes that may consist of foil-lined interiors, such as aluminum or an alloy. Other containers include test tubes, vials, flasks, bottles, syringes, and the like. Containers may have a sterile access port, such as a bottle having a stopper that can be pierced by a hypodermic injection needle. Other containers may have two compartments that are separated by a readily removable membrane that upon removal permits the components to mix. Removable membranes may be glass, plastic, rubber, and the like.

In certain embodiments, kits can be supplied with instructional materials. Instructions may be printed on paper or other substrate, and/or may be supplied as an electronic-readable medium, such as a floppy disc, mini-CD-ROM, CD-ROM, DVD-ROM, Zip disc, videotape, audio tape, and the like. Detailed instructions may not be physically associated with the kit; instead, a user may be directed to an Internet web site specified by the manufacturer or distributor of the kit.

Compositions and methods described herein utilizing molecular biology protocols can be according to a variety of standard techniques known to the art (see, e.g., Sambrook and Russel (2006) Condensed Protocols from Molecular Cloning: A Laboratory Manual, Cold Spring Harbor Laboratory Press, ISBN-10: 0879697717; Ausubel et al. (2002) Short Protocols in Molecular Biology, 5th ed., Current Protocols, ISBN-10: 0471250929; Sambrook and Russel (2001) Molecular Cloning: A Laboratory Manual, 3d ed., Cold Spring Harbor Laboratory Press, ISBN-10: 0879695773; Elhai, J. and Wolk, C. P. 1988. Methods in Enzymology 167, 747-754; Studier (2005) Protein Expr Purif. 41(1), 207-234; Gellissen, ed. (2005) Production of Recombinant Proteins: Novel Microbial and Eukaryotic Expression Systems, Wiley-VCH, ISBN-10: 3527310363; Baneyx (2004) Protein Expression Technologies, Taylor & Francis, ISBN-10: 0954523253).

Definitions and methods described herein are provided to better define the present disclosure and to guide those of ordinary skill in the art in the practice of the present disclosure. Unless otherwise noted, terms are to be understood according to conventional usage by those of ordinary skill in the relevant art.

In some embodiments, numbers expressing quantities of ingredients, properties such as molecular weight, reaction conditions, and so forth, used to describe and claim certain embodiments of the present disclosure are to be understood as being modified in some instances by the term “about.” In some embodiments, the term “about” is used to indicate that a value includes the standard deviation of the mean for the device or method being employed to determine the value. In some embodiments, the numerical parameters set forth in the written description and attached claims are approximations that can vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the present disclosure are approximations, the numerical values set forth in the specific examples are reported as precisely as practicable. The numerical values presented in some embodiments of the present disclosure may contain certain errors necessarily resulting from the standard deviation found in their respective testing measurements. The recitation of ranges of values herein is merely intended to serve as a shorthand method of referring individually to each separate value falling within the range. Unless otherwise indicated herein, each individual value is incorporated into the specification as if it were individually recited herein.

In some embodiments, the terms “a” and “an” and “the” and similar references used in the context of describing a particular embodiment (especially in the context of certain of the following claims) can be construed to cover both the singular and the plural, unless specifically noted otherwise. In some embodiments, the term “or” as used herein, including the claims, is used to mean “and/or” unless explicitly indicated to refer to alternatives only or the alternatives are mutually exclusive.

The terms “comprise,” “have” and “include” are open-ended linking verbs. Any forms or tenses of one or more of these verbs, such as “comprises,” “comprising,” “has,” “having,” “includes” and “including,” are also open-ended. For example, any method that “comprises,” “has” or “includes” one or more steps is not limited to possessing only those one or more steps and can also cover other unlisted steps. Similarly, any composition or device that “comprises,” “has” or “includes” one or more features is not limited to possessing only those one or more features and can cover other unlisted features.

All methods described herein can be performed in any suitable order unless otherwise indicated herein or otherwise clearly contradicted by context. The use of any and all examples, or exemplary language (e.g. “such as”) provided with respect to certain embodiments herein is intended merely to better illuminate the present disclosure and does not pose a limitation on the scope of the present disclosure otherwise claimed. No language in the specification should be construed as indicating any non-claimed element essential to the practice of the present disclosure.

Groupings of alternative elements or embodiments of the present disclosure disclosed herein are not to be construed as limitations. Each group member can be referred to and claimed individually or in any combination with other members of the group or other elements found herein. One or more members of a group can be included in, or deleted from, a group for reasons of convenience or patentability. When any such inclusion or deletion occurs, the specification is herein deemed to contain the group as modified thus fulfilling the written description of all Markush groups used in the appended claims.

Citation of a reference herein shall not be construed as an admission that such is prior art to the present disclosure.

Having described the present disclosure in detail, it will be apparent that modifications, variations, and equivalent embodiments are possible without departing the scope of the present disclosure defined in the appended claims. Furthermore, it should be appreciated that all examples in the present disclosure are provided as non-limiting examples.

### EXAMPLES

The following non-limiting examples are provided to further illustrate the present disclosure. It should be appreciated by those of skill in the art that the techniques disclosed in the examples that follow represent approaches the inventors have found function well in the practice of the present disclosure, and thus can be considered to constitute examples of modes for its practice. However, those of skill in the art should, in light of the present disclosure, appreciate that many changes can be made in the specific embodiments that are disclosed and still obtain a like or similar result without departing from the spirit and scope of the present disclosure.

### Example 1: Determination of Mechanical Memory of Migrating Cells of Past Matrix Stiffness

The following example describes experiments describing fabrication of devices that can allow for the determination of mechanical memory of collectively migrating cells.

Fabrication of 2D Substrates with Heterogeneous Stiffness

Specifically, a polyacrylamide (PA) precursor solution of acrylamide (monomer-AA), bisacrylamide (crosslinker-BIS), and azoebis (a photoinitiator) was polymerized under UV light through a photomask (see e.g., FIG. 2A). In this method, variation in grayscale percentages of the mask proportionally limits UV crosslinking of PA and allows spatial control over PA stiffness. This photomask creates a primary ECM region of defined stiffness (Ep) and a surrounding secondary ECM of dissimilar stiffness (Es). The regional stiffness maps are generated through Atomic Force Microscopy (AFM). The cells are seeded in the primary ECM, confined by a polymer mold (a PDMS stencil). Next, the PDMS stencil is lifted and the cells are allowed to invade the secondary ECM. Here, Ep will be varied between 0.5-25 kPa, corresponding to the primary tumor stiffness, and the Es, corresponding to the secondary tissue stiffness, will be kept constant at 5 kPa for the first study. These studies will be repeated for Es=1, 10, 15, 20, and 25 kPa. To further expand the range of stiffness to between 0.08-120 kPa, different ratios of monomer-AA (3-15%) and crosslinker-BIS (0.05-1.2%) will be combined using a microfluidic mixing setup (see e.g., FIG. 2B) and polymerize these PA solutions of varied ratios in distinct regions of a continuous substrate. For Traction Force Microscopy (TFM), fluorescently-labeled beads will be mixed in the PA solution. Substrates will be coated with collagen type 1, because breast tissue is known to be collagen-rich. Experiments will be repeated for varying protein densities and types (e.g., elastin, laminin, fibronectin, and other collagen types) to investigate whether cellular plasticity and memory depend on ECM protein properties.

Cell Lines

These studies start with MCF10A cells, a mammary epithelial cell line. While it is not a tumorigenic line, MCF10A was chosen given its customizability in terms of molecular perturbations and oncogenic manipulations. To selectively induce known behaviors of cancer cells, +Rho, +YAP, −YAP, and −αcatenin, versions of MCF10A were developed. Next, plasticity and memory of various breast cancer cells (e.g., MCF7, MDA-MB231, and Eph4Ras) and MCF10A variants of specific oncogenic lesions (e.g., MCF10DCIS, MCF10AT and with overexpressed H-Ras, ErbB2, or 14-3-3ζ) are assessed. See Example 2 for studies with MCF7, MCF10A, and A431 cell lines.

Measurements in Secondary ECM

First, the leading edge migration speed and velocity maps (using PIV) of a cell sheet migrating from the primary (Ep) to the secondary (Es) ECM will be measured by conducting time-lapse microscopy over ˜4 days. The samples will be fixed and stained for known mechno-regulators of cell behavior, such as actomyosin components (F-actin, pMLC) and focal adhesion proteins (vinculin, paxillin, pFAK). In addition, the expressions of YAP, TWIST1, ERK, and selected proteins of the Rho/Rac family will be measured because of their known roles in perturbing epithelial morphogenesis in breast cancer. Specifically, TWIST1 and YAP translocate into the nucleus for various breast cancer cells seeded on stiff matrices, while they remain cytoplasmic on soft matrices. Along with TWIST1 and YAP, ERK activation and ROCK-generated contractility are tied to the clustering and activation of integrins at the cell-ECM interface. Measuring the gradients in expression of these molecules across changing ECM stiffness on a single substrate will demonstrate the rate of stiffness-dependent cellular adaptation to new environments, i.e., cellular plasticity. Immunostaining and confocal microscopy will be used to visualize these molecules and quantify molecular expressions through image analysis. Further molecular screening will be conducted by evaluating mRNA expression through PCR and SDS-PAGE. In order to conduct molecular analysis of cells after travelling a defined distance (ds) through the secondary ECM, the cells will be collected through flow channels (explained in more detail further ahead; see e.g., FIG. 8) attached at distance “ds” from the primary ECM interface. These measurements will be repeated in all devices.

In samples in which “Eₚ” is greater than 5 kPa, the epithelial cells might undergo stiffness-dependent EMT, as has been reported earlier and confirmed here (see e.g., FIG. 6), and migrate into the secondary ECM as small clusters or single cells. In those cases, single/clustered cell migration speeds versus primary ECM stiffness will also be measured and plotted. To better understand how EMT signatures change over distance, known EMT markers (e.g., E-cadherin, vimentin, N-cadherin, and β-catenin) will be visualized for all ECM conditions. In further experiments, TGF-β, a growth factor known to induce EMT, will be added during cell seeding and the variation of EMT markers will be plotted as function of the primary ECM stiffness “Eₚ” and the distance “dₛ”.

The displacement of beads embedded underneath the PA surface will be combined with Fourier Transform Traction Force Cytometry to perform TFM and visualize variations in regional cellular force maps from primary to secondary ECMs. If the hypothesis of cellular memory holds true, the cells that started on stiff primary ECM will continue to generate higher forces even after their migration into the soft secondary ECM regions. Preliminary data-collective migration speed remembers past stiffness. To test the proposed methodology, a 2D substrate of two different stiffness regions—a primary ECM of 25 kPa stiffness on the left and a secondary ECM of 1 kPa stiffness on the right—were fabricated as illustrated in FIG. 4A. Next, a colony of MCF10A cells were cultured on the primary ECM and allowed it to migrate into the secondary ECM. The migration speed of the leading edge (vs) after its arrival in the secondary ECM was calculated. For comparison, the experiments where stiffness of primary and secondary ECMs was kept identical (Es=1 kPa) were repeated. It was found that the cells that were first cultured on the stiffer primary ECM migrated faster after arriving in a soft secondary ECM (see e.g., FIG. 4B), as compared to those initially cultured on a soft ECM. It is proposed that these differences in the speed of cells that originated on different ECMs persist due to the mechanical memory of past environments stored in the cells. These observations reveal a novel concept that migrating cells do not exhibit pure plasticity, rather they tend to store mechanical memory of their past environments. These experiments were repeated with MCF10A-α-catenin-knockdown (MCF10A-AC), with disabled cell-cell junctions, and found that dependence of speed vs on primary ECM stiffness, Ep, increased even further. These results indicate that abrogation of cell-cell junctions, which is a hallmark of cancer cells, amplifies ECM-sensitivity and enhances the memory of past mechanical ECMs. See Example 2 for additional measurements in secondary ECM.

Quantifying Cellular Plasticity and Memory

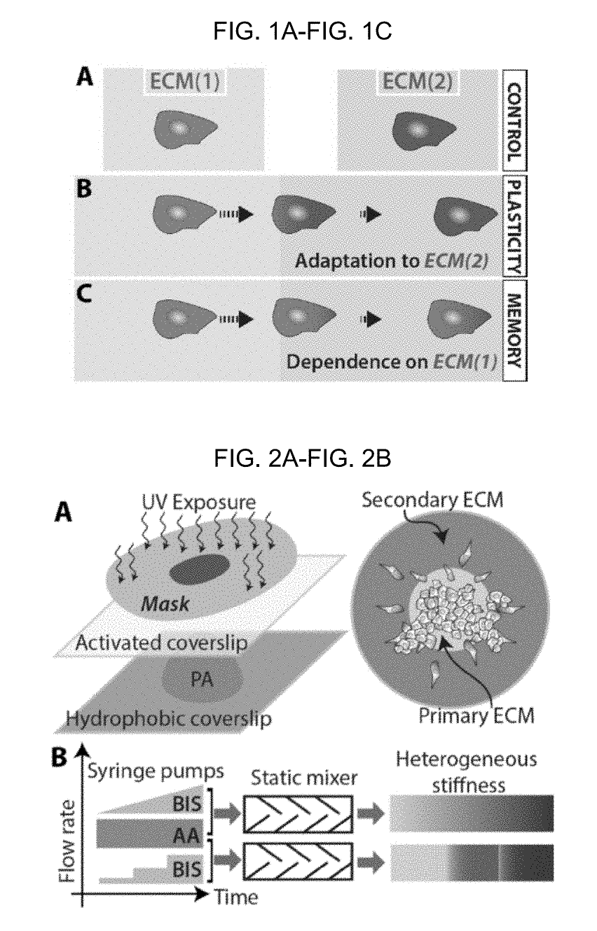

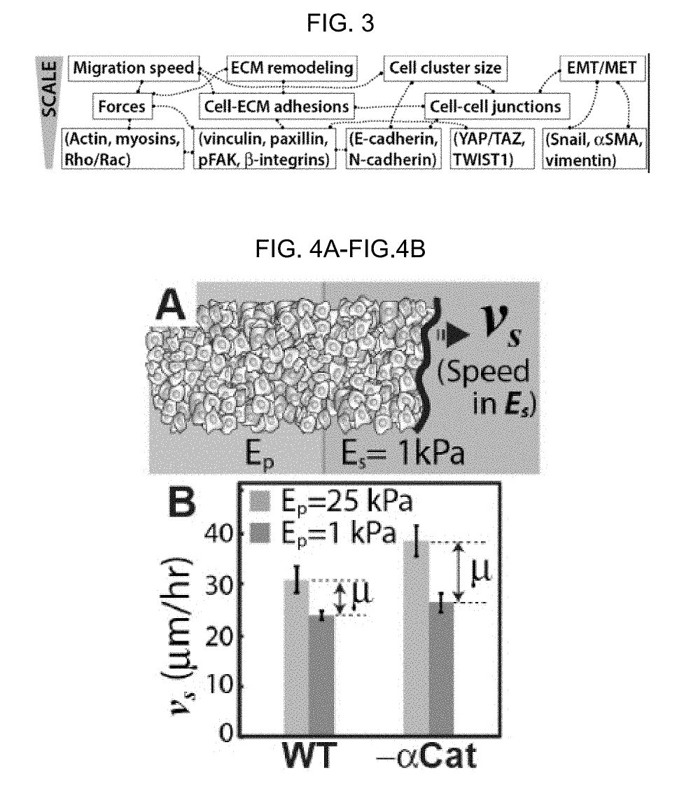

In all experiments, the degree of cellular plasticity and memory will be quantified according to how a given cell behavior changes with respect to time or distance across ECMs of dissimilar properties. Here, the studied cell behaviors could be the epithelial/mesenchymal states of cells, the degree of EMT or MET, activation levels of certain subcellular molecular pathways, and various modes of cell migration (amoeboid, mesenchymal, clustered, or collective). The ECM properties in different regions of a given device can vary in terms of stiffness (or, porosity, degradability, confinement, microstructure, ECM proteins, and the co-cultured cell types—to be introduced ahead). Consider that a cell behavior, labeled as “c1”, corresponds to cells cultured solely on ECM “E1”, and behavior “c2” of cells cultured separately on ECM “E2” (see e.g., FIG. 1). Now, if cells cultured on ECM “E1” move to ECM “E2”, the ‘cellular plasticity’ is defined by the time taken or distance traveled by the cells to transition from “c1” to “c2”. The ‘cellular memory’ will be defined as the difference, “μ=c1−c2”, for cells in ECM “E2” with respect to time or distance. Larger values of “p” would indicate lesser cellular plasticity and greater retention of memory of past environments. See Example 2 for additional studies regarding how past matrix stiffness primes epithelial cells and regulates their future collective migration through a mechanical memory.

Collective Invasion in Compliant Microchannels

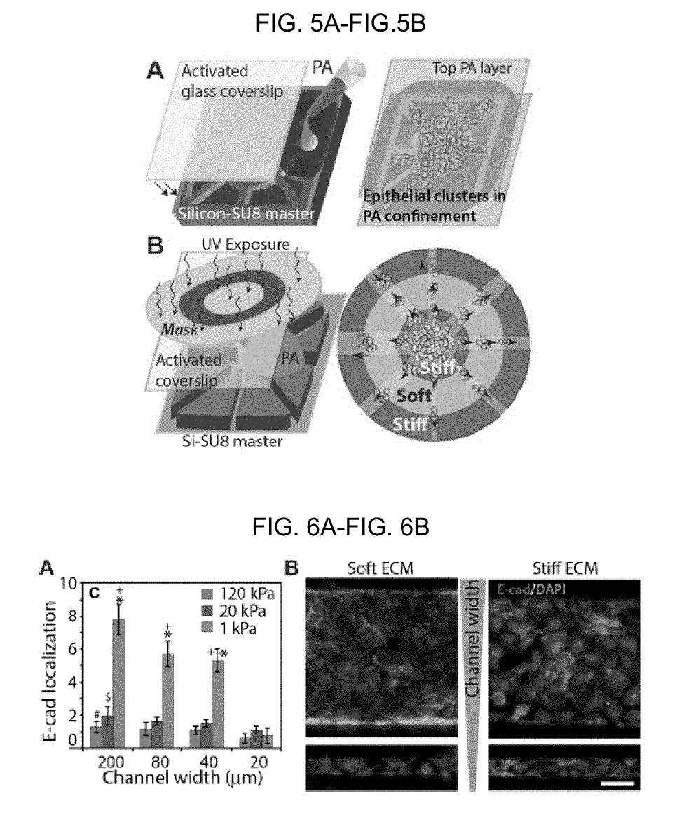

To introduce 3D confinement around cells, the PA gel in the secondary ECM will be molded into microchannels by adopting a ‘PA channels’ methodology. A coverglass with a layer of matching PA gel will be adhered (using Cell-Tak adhesive) on top of the channels to close the device (see e.g., FIG. 5A). By fabricating channels within the photo-polymerization based framework described earlier (as in FIG. 2), different regions of the same substrate will have independently tunable stiffness and confinement (channel widths 20 μm to 200 μm), FIG. 5B, which has not been achieved before. Using this platform, interactions among various mutant cells during collective migration of heterogeneous cancer cell populations through ECMs of varying stiffness and confinement will be studied. In confined ECMs, the enhanced cell-ECM interactions might weaken cell-cell junctions and lead to greater de-clustering, EMT, and invasion over time. In addition to the measurements noted earlier, quantification of the size and morphology of cell clusters as they traverse through channels will be performed (see e.g., Example 2). Thus, the stiffness and confinement of past environments may dictate the size and speed of cancer cell clusters at future time points.

EMT Due to Stiffness and Confinement

To understand how epithelial cell clusters might respond to ECM confinement, MCF10A cells were cultured in PA channels of varying stiffness and confinement, and measured various EMT markers (Ecad, vimentin, cell morphology). These experiments revealed that EMT occurs more readily in confined environments (see e.g., FIG. 6). Surprisingly, confinement induced EMT even in cell clusters surrounded by soft matrices, which otherwise protect against EMT in unconfined environments. Thus, heterogeneity in ECM confinement and stiffness over the tumor cell invasion trajectory is likely to complicate the ability of cells to adapt to new environments.

Fabrication of Degradable 3D ECMs in Multiple Zones

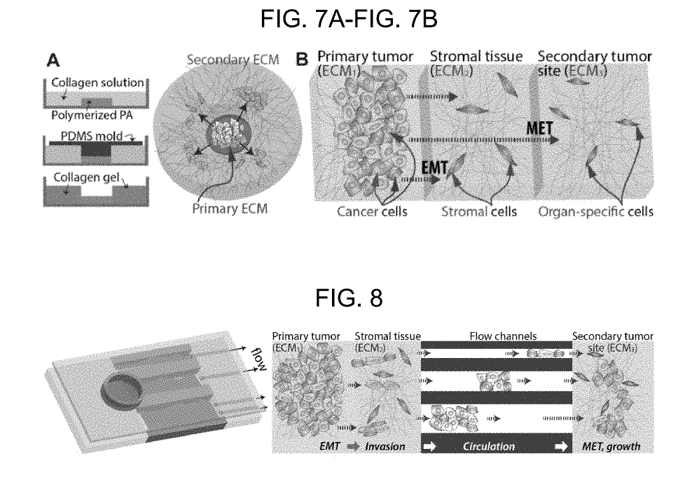

First, cells cultured on a PA-based primary ECM will be surrounded by a 3D collagen ECM in a well-like setup (see e.g., FIG. 7, FIG. 31). A PA gel of defined stiffness, with ˜5 mm diameter and ˜100 μm thickness, will be polymerized in the center. Next, a PDMS stencil will cover the PA gel and the collagen solution will be gelled around it. The PA substrate will be coated with an ECM protein (collagen at first), and a cell colony will be seeded. Thus, cells cultured on a primary ECM of defined stiffness will transmigrate into a secondary ECM of dissimilar dimensionality, fiber structure, and stiffness.

In the next iteration of the device, the existing framework of 2D-to-3D cancer cell invasion will be modified such that the primary ECM is also composed of a 3D collagen gel of mechanical properties distinct from those of the surrounding ‘secondary’ collagen ECM. This will be achieved by first polymerizing a surrounding secondary collagen gel, and, subsequently, filling the central ‘primary’ region with a mixture of cancer cells and collagen. Such a design will create a more mechanically realistic situation in which cancer cells embedded in a 3D matrix represent an ‘engineered tumor’, which is surrounded by a secondary 3D ECM of dissimilar mechanical properties. To capture the complex ECM remodeling during stromagenesis, apart from varying collagen concentration, fiber width and crosslinking density will also be manipulated for a given collagen content by manipulating the pH of the collagen solution and enabling lysyl oxidase (LOX)-based photo-crosslinking, respectively. These changes in fiber microstructure will alter the known nonlinear material behavior of collagen.

Finally, to better mimic the biomechanical conditions of the tumor microenvironment, cancer cells of varying oncogenic mutations will be co-cultured to create a ‘tumor-like’ heterogeneous population in the primary ECM. In the secondary ECM, non-tumor cells such as fibroblasts, macrophages, and adipocytes will be included to better mimic the stromal tissue. The cancer cells exiting out of the secondary ECM will be collected for molecular analysis. The interaction of cancer cells with other cells might affect their ability to adapt to new environments and alter cancer cell plasticity and memory measured thus far.

Measurements and Interpretation of Results

As described in the experiments above and Example 2, measurement of the migration speed and overall invasiveness of cells (single or clustered) migrating from the primary to the secondary ECM of different mechanical (stiffness, dimensionality, fibrosity) and biological (cell types and ECM protein) properties will be performed. Expressions of subcellular molecular pathways that are known to be mechano-sensitive will also be measured. Changes in memory measurements, compared to those in 2D devices, will be attributed to the new ‘in vivo-like properties’, e.g., dimensionality, collagen architecture, and co-cultured cells. Organization of collagen fibers, remodeled and degraded due to cell invasion, will be measured through Second harmonic generation (SHG) imaging of collagen and compared for varying primary ECMs. As indicated before, cancer cell memory will be assessed by how cancer cells cultured on differing primary ECMs respond differently to a given secondary ECM. In contrast, cancer cell plasticity will be assessed by how quickly cells ‘forget’ their previous ECM and only respond to the properties of their current secondary ECM.

Fabrication of Devices with Varying Distance Between Primary and Secondary ECMs

The trajectory of tumor invasion involves long-distance circulation of cancer cells through blood vessels before reaching metastatic sites away from the primary tumor. The in-vitro matrix platforms proposed thus far capture how cancer cells adapt to new environments in close vicinity to the primary tumor. In order to assess how the plasticity and memory of cancer cells change with longer distances from the primary tumor, modify the existing platform by connecting the primary and the secondary ECMs through ‘flow channels’ of varied length (see e.g., FIG. 8) is performed. First, a PDMS-based microfluidic device will be constructed where two compartments will be connected through channels of at least 30 μm height and variable length (2-50 mm) and width (20-500 μm). The curvature in channel geometry attempts to mimic the tortuous circulation of cancer cells. Next, a collagen gel corresponding to the biomechanical properties for the secondary ECM at the metastatic tissue site will be polymerized in the right compartment. The composition of the secondary matrix can be modified to better mimic the metastatic sites by mixing different ECM proteins and relevant cell types. The left compartment will contain a two-layered ECM system: (1) primary ECM of cancer cells mixed with collagen of biomechanical properties corresponding to the primary tumor, and (2) a stromal ECM with dissimilar collagen properties and relevant cell types (fibroblasts, macrophages). Finally, the device will be sealed and a left-to-right flow in the channels will be established such that the cells exiting from the primary and stromal ECMs will flow through the channels of defined length and reach the distant secondary ECM. The length of the channels will dictate the distance between the primary tumor and the secondary metastatic site. The varied channel lengths will allow us to assess the limits of cellular memory. The variation in channel width will allow evaluation of cellular plasticity and memory based on the size of cell groups escaped from the tumor. Here, wide channels are likely to carry sheets and big chunks of cells, while narrower channels might transmit smaller clusters or streams of cells, much like the in vivo heterogeneity.

Measurement of Cancer Cell Memory Over Long Distances

In addition to the measurements indicated previously, the degree of proliferation of cancer cells in the distant secondary ECM will be measured, which corresponds to the ability of cancer cells to grow secondary tumors at distant locations—a hallmark of cancer metastasis. For a constant channel length (distance between primary and secondary ECM), it will be assessed if cells originated from different primary tumor environments show differing abilities to grow in the same secondary ECM, which would indicate a persistent ‘environmental memory’ possessed by cancer cells. Next, channel length will be varied for a given combination of primary and secondary ECMs, and how cellular plasticity and memory change with the distance traveled by cancer cells will be measured. These measurements reveal phenomenological differences between metastases to ‘near’ versus ‘far’ locations relative to the primary tumor.

Modular Polyacrylamide Substrates

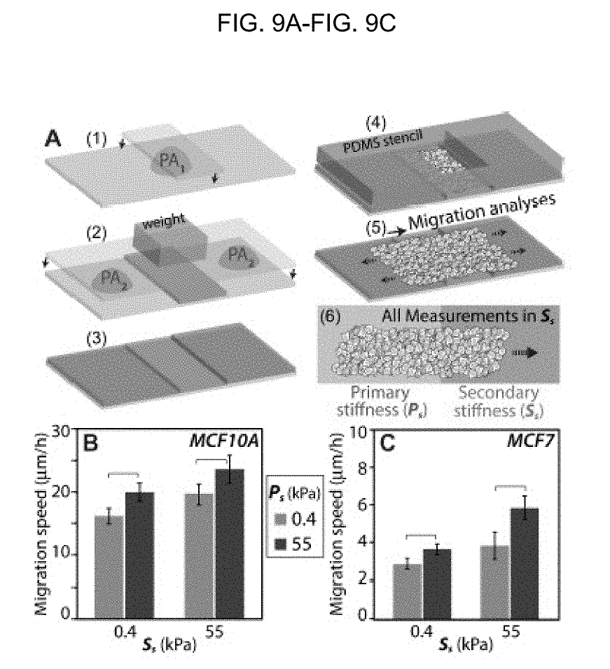

Contiguous polyacrylamide gels with distinct modules were fabricated through a step-by-step polymerization of PA solutions of defined compositions. Precursor solutions containing the acrylamide:bis-acrylamide (A:B) percentages of 5:0.05% or 12:0.6%, corresponding to PA gels of elastic moduli of 0.4 and 55 kPa, were mixed with 0.5% Ammonium Persulphate (APS), 0.05% Tetramethylethylenediamine (TEMED). Red fluorescent carboxylate-modified beads of 200-nm diameter were added at 0.1% concentration in the stiff PA precursor solution to identify the interface between dissimilar ECM modules. Next, a volume of PA precursor solution sufficient to achieve a gel thickness of 100 μm was dispensed on the coverslip and sandwiched between two glass slides to confine the spreading of the PA droplet in each module of the substrate. This step was repeated for all three modules (see e.g., FIG. 9A) to fabricate the entire modular PA (mPA) hydrogel substrate. After polymerization, mPA gels were incubated with 0.05 mg/ml of rat-tail collagen type I (Santa Cruz Biotechnologies) overnight at 4° C.

Collective Cell Migration Assay

Human mammary epithelial non-tumorigenic MCF10A cells were cultured in DMEM-F12 (GE Healthcare Life Sciences), supplemented with 5% horse serum (Invitrogen), 20 ng/mL epidermal growth factor (EGF, Miltenyi Biotec Inc), 0.5 mg/mL hydrocortisone (Sigma-Aldrich), 100 ng/mL cholera toxin (Sigma-Aldrich), 10 μg/mL insulin (Sigma-Aldrich), and 1% (v/v) penicillin-streptomycin (Sigma-Aldrich). Tumorigenic mammary epithelial MCF7 cells were grown in DMEM (Sigma-Aldrich) containing 10% fetal bovine serum (FBS), 1% (v/v) penicillin-streptomycin (Sigma-Aldrich), and 1% non-essential amino acids (0.1 mM). MCF10A cells expressing constitutively active Rho were generated using a QL activating mutant Rho subcloned into a pFLRu vector. PDMS stencil was designed and fabricated with a rectangular opening in the center, restricting the culture of epithelial monolayer within the central module (primary ECM) of mPA substrate. PDMS stencils were treated with 1 mg/ml of BSA to avoid cell adhesion and press-bonded on to the PA gels. Cell suspension with 2×10⁴ cells was dispensed into the PDMS reservoir covering the primary ECM region and left to grow for 18 h at 37° C. in a 5% CO₂ humidified incubator. After this incubation period, the PDMS stencil was lifted off to allow cell sheet to migrate into the adjoining secondary ECM. Additional media was added to each well. Time-lapse microscopy was initiated 2 h after removing the PDMS stencil and all the migration experiments were carried out at 37° C. and in 5% CO₂ environment within an incubator staged over the microscope.

Time-Lapse Microscopy and PIV Analysis

Time-lapse imaging was carried out in the phase contrast on an inverted microscope (Zeiss Cell Observer) equipped with an incubator capable of maintaining an environment with 37° C. temperature and 5% CO₂ level. Images were acquired with a 10× objective. Two successive images of the same field were taken at 1 h time interval. Motion of the leading edge of the cell monolayer was manually tracked by recording the position of cell border using a homemade macro in ImageJ. Subsequently, coordinates of the leading edge were imported into a custom-made program in MATLAB and its advancement was calculated by averaging the distance of each point on the leading edge from the primary-secondary ECM interface. Leading edge speed was defined as the ratio of the leading edge advanced distance and the time course of migration. Monolayer velocity fields were computed using custom-made PIV software written in MATLAB. In PIV analysis, each image was broken down into defined pixel windows for comparison. To reduce the systematic biases in subpixel resolution, PIV was iteratively implemented (up to 4 iterations). Displacement and velocity (displacement/time interval) vectors were calculated by comparing the displacement of a window between two successive images. The velocity field was expressed in μm/h. PIV analysis yielded two components of velocity at each point (i,j), namely lateral (uij, perpendicular to the direction of group migration) and axial (vij, along the direction of group migration). After obtaining the velocity vectors, cell alignment and cell-cell coordination were evaluated in terms of correlation length and order parameter. The order parameter was defined as the cosine of the angle that the velocity vector makes with the principal direction of migration. The order parameter varies from +1 (for velocity vectors parallel to the strip and directed along the direction of migration of the cell sheet) to −1 (for vectors that are directed opposite to the direction of migration of the cell front) through 0 (for vectors aligning perpendicular to the direction of the monolayer migration).

Immunofluorescence and Confocal Microscopy

After day 5, cells in the migration assay were rinsed with cold 1× Phosphate buffered saline (PBS) for 2-3 minutes and fixed in 4% Paraformaldehyde (PFA) at room temperature (RT) for 10 minutes. After washing again with PBS, cells were incubated with 1% bovine albumin serum (BSA) (EMD millipore) overnight at 4° C. Next, cells were washed with PBS for 30 min, and incubated in primary antibody solution prepared in 1% BSA, and stored overnight at 4° C. Samples were washed and incubated with appropriately matched secondary antibodies for 1 hour at RT. After thoroughly rinsing the substrates with PBS, 1:250 10 mg/mL DAPI (Santa Cruz) was added for 30 min at RT. Finally, substrates were rinsed again with PBS and stored at 4° C. before imaging. Images were recorded at RT using a laser-scanning confocal microscope (Ziess LSM 730; Carl Ziess Microlmaging, Germany) at 20× objective, and confocal stacks were acquired at 1 μm interval. Experiments were performed in triplicates, and the images used for analysis were selected randomly from 10-15 fields for each condition.

Subcellular YAP Localization

Captured z-stacks were imported to ImageJ (NIH) as LSM files, and the stacks were projected with maximum intensity setting. To quantify the subcellular localization of YAP, cells were visually checked for the nuclear inclusion/exclusion and the percentage of each type of YAP localization, which was calculated by finding the number of cells representing the corresponding YAP localization category (nuclear, cytoplasmic, or intermediate).

Fabrication of a Contiguous Substrate with Distinct Regions of Defined Stiffness

To precondition epithelial cells on a given ECM and track their subsequent collective migration on an adjoining ECM of dissimilar stiffness, a PA substrate was fabricated through a step-by-step polymerization of two different PA compositions (see e.g., FIG. 9A). This modular-PA (mPA) hydrogel system was divided into three regions of either 0.4 kPa (soft) or 55 kPa (stiff) elastic moduli. A PDMS stencil for culturing an epithelial monolayer restricted within the central section of the mPA gel was also designed, which is referred to as the ‘primary’ ECM. After 2 days of preconditioning of cells on the primary ECM, the PDMS stencil was removed to enable collective migration of the cell sheet into the surrounding ‘secondary’ ECM of a different stiffness. In this system, epithelial cells seamlessly move across a contiguous substrate composed of mechanically distinct regions, whose stiffness can be tuned over two orders of magnitude. In this system, it is now possible to study the effect of past ECM stiffness on future cell behavior without having to detach and re-culture cells on a new substrate. In order to isolate the immediate influence of primary ECM stiffness on cell behavior, all cellular measurements are performed in the secondary ECM only. Comparison of cell behavior on a given secondary ECM with respect to varying primary ECM stiffness would reveal whether migratory epithelial cells store mechanical memory of their past mechano-regulated state.

Leading Edge Migration Depends on the Past ECM Stiffness

To understand how ECM stiffness alone influences collective cell migration, MCF10A human mammary epithelial cells were cultured on either homogeneously soft or stiff ECM for 2 days using the PDMS stencil described above and then allowed the cell sheet to migrate onto the surrounding ECM of the same stiffness. Time-lapse microscopy was performed between days 3-5 to record the movement of the leading edge. The leading edge of the cell sheet was found to migrate ˜50% faster on the stiff ECM compared to the soft ECM (see e.g., FIG. 9B), consistent with previous findings (7). To assess the effect of past ECM stiffness on collective cell migration, MCF10A cells were cultured on stiff primary ECM (55 kPa) for 2 days and then allowed cells to migrate onto a soft secondary ECM (0.4 kPa). Leading edge migration speeds were compared for cells preconditioned on soft and stiff primary ECMs as they migrated onto given soft secondary ECM. After arriving on a soft secondary ECM, cells that originated from a stiff primary ECM migrated ˜25% faster than those initially cultured on a soft primary ECM (see e.g., FIG. 9B). This enhanced leading edge migration could be attributed to the stiffness-sensitive state of cells attained in the stiff primary ECM—a property referred to as the ‘mechanical memory’ of collectively migrating cells. Next, cells were preconditioned on a soft primary ECM before migrating onto a stiff secondary ECM. When compared to the corresponding control case of a homogeneously (primary and secondary) stiff ECM, cells originating from a soft primary ECM migrated ˜20% slower than those initially cultured on a stiff primary ECM.

Next, the mechanical memory-dependent collective cell migration of the non-tumorigenic MCF10A cells were compared with tumorigenic, but non-metastatic MCF7 cells. Analysis of the leading edge migration demonstrated that MCF7 cells migrated significantly slower than MCF10A cells. However, the effect of memory still persisted in these cells, i.e. preconditioning by a stiff primary ECM enhanced leading edge migration while initial culture on a soft primary slowed migration in a dissimilar secondary ECM (see e.g., FIG. 9C).

Migratory Cells Store Mechanical Memory of Past ECM Stiffness Through YAP Activity

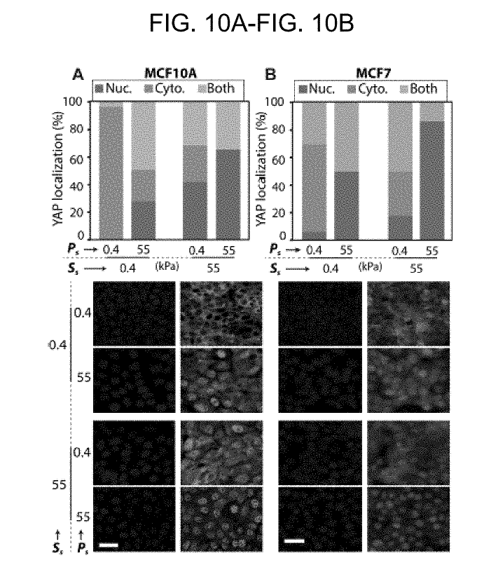

The leading edge cell migration analysis across dissimilar matrices (see e.g., FIG. 9) indicates that the ECM stiffness-dependent state of cells may persist after their transmigration onto a new ECM. It was hypothesized that the mechanosensitive activation of the mechanotransduction signaling in the primary ECM might be responsible for sensing and storing mechanical memory of ECM stiffness. The nuclear translocation of YAP has been identified as a sensor of ECM stiffness. Thus, a variation in YAP localization within the cells was investigated as they migrated across ECMs of dissimilar stiffness. Through immunofluorescence analysis, three distinct patterns of the intracellular YAP localization—nuclear, cytoplasmic, and intermediate (both) were identified. First, experiments with MCF10A cells were performed and quantified the percentage emergence of each form of cellular YAP localization. When cultured and allowed to migrate solely on stiff substrates for 5 days, ˜65% MCF10A cells in the monolayer exhibited predominantly nuclear YAP localization and no cells had exclusively cytoplasmic YAP expression (see e.g., FIG. 10A). Consistent with previous finding, in MCF10A cells on homogenously soft substrates, YAP expression was predominantly cytoplasmic (see e.g., FIG. 10A).

However, when an epithelial sheet was first cultured on a stiff substrate and YAP subcellular localization determined in cells on a soft secondary ECM, only ˜20% cells showed cytoplasmic only YAP localization even after arriving to a soft ECM (see e.g., FIG. 10A). Thus, nuclear accumulation of YAP due to the stiffer past ECM largely persists even after the cells migrated into the adjoining softer region. In cells preconditioned on a soft ECM, only ˜25% cells on the stiff secondary ECM retained the cytoplasmic YAP localization, compared to no cells with cytoplasmic localization of YAP when migrating on homogenously stiff primary and secondary substrates (see e.g., FIG. 10A). In both of cases of cell migration across dual-stiffness regions (e.g., high to low and low to high), the ECM stiffness-dependent subcellular localization of YAP in the primary region persisted after cells migrate into secondary environments (see e.g., FIG. 10A). Thus, cellular mechano-sensation of ECM stiffness through YAP localization could be the key mechanism for storing mechanical memory in migratory cells.

These measurements were repeated in MCF7 cells (see e.g., FIG. 10B) and found that preconditioning on a stiff primary ECM led to ˜50% cells with nuclear YAP on the soft secondary ECM. Similarly, less than 20% cells on a stiff secondary ECM had nuclear YAP localization due to preconditioning on a soft primary ECM. Comparison of these numbers with those for MCF10A cells indicated that in MCF7 cells YAP nuclear localization could have greater influence upon mechanical memory.

Stiffer Primary ECM Predicts a More Correlated Collective Migration

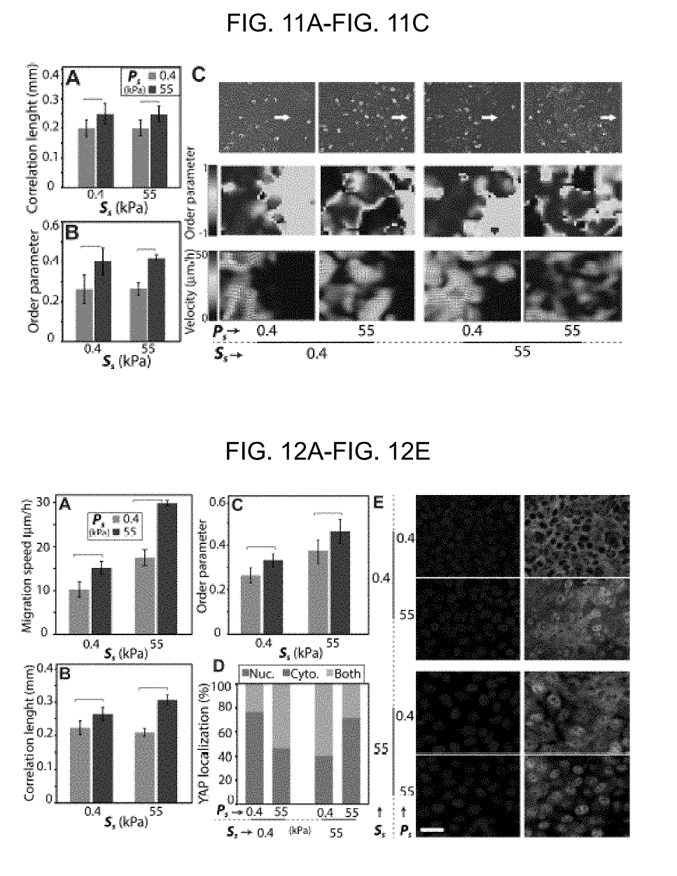

Using PIV analysis of phase contrast images of the migrating cell sheet over time, cellular motions within the epithelial monolayer were examined (see e.g., FIG. 11C). The correlation length was quantified as the distance over which velocity vectors of neighboring cells correlate with one another. It was determined that the correlation length for cells migrating on a homogeneously stiff ECM was ˜0.25 mm, which is ˜25% higher than its value measured on a purely soft ECM (see e.g., FIG. 11A). Thus, higher ECM stiffness enables larger portions of the cell sheet to migrate in a coordinated fashion, which is consistent with previous findings. When preconditioned on a stiff primary ECM, cells migrated on a soft secondary ECM with ˜0.25 mm correlation length, the same as the value measured on a homogeneously stiff ECM. However, cells preconditioned on a soft primary ECM migrated in the adjoining stiff secondary ECM with a lower correlation length of ˜0.2 mm, similar to the case of a homogeneously soft ECM. Thus, correlation lengths in both soft and stiff secondary ECMs are dictated by the primary ECM stiffness. The order parameter, defined as the angle between the velocity vectors of cells within the sheet and the principal direction of leading edge migration, was computed. It was found that the order parameter of the collective migration on a homogeneously stiff ECM was ˜58% higher than on a soft ECM (see e.g., FIG. 11B, FIG. 11C), which indicates that higher stiffness leads to a more ordered collective migration. Consistent with the framework of the mechanical memory during collective migration described thus far, the order parameter corresponding to a stiff primary ECM remained high (0.4, similar to the value on purely stiff ECM) even through it was measured for the cells sheet migrating in the soft secondary ECM. Conversely, after preconditioning on a soft ECM, the cell sheet migrated with a lower order parameter (0.26, similar to the value on purely soft ECM) even after arriving in a stiff secondary ECM. Thus, both the correlation length and the order parameter are regulated by the primary ECM stiffness (see e.g., FIG. 11).

Activation of RhoA Enhances the Mechanical Memory Dependent Collective Migration

Cellular mechanosensing is crucially tied to RhoA GTPase activity and the associated regulation of actin-myosin contractility and cell-ECM adhesions. Additionally, since the ability of cells to sense ECM stiffness is a crucial prerequisite for storing the mechanical memory in cells, it was sought to understand how RhoA activation might influence memory. To this end, a MCF10A cell line expressing GTPase-deficient constitutively active RhoA (Rho-CA) was generated and repeated measurements on the mPA scaffolds. It was found that Rho-CA cells migrated faster on a homogeneously stiff ECM as compared to MCF10A wildtype (WT) cells (see e.g., FIG. 12A). On soft the ECM, Rho-CA cells migrated slower than WT cells. Thus, RhoA activation amplified the migration speed differences between soft and stiff ECMs. Across dual-stiffness mPA substrates, Rho-CA cells first cultured on the stiff region migrated faster on the soft secondary ECM compared to those arriving from a soft primary ECM (see e.g., FIG. 12A), which is consistent with the behavior observed in WT cells (see e.g., FIG. 9B). On the stiff secondary ECM, Rho-CA cells preconditioned on a soft primary ECM migrated slower than those from a stiff primary ECM (see e.g., FIG. 12A), which also matches the trends for WT cells (see e.g., FIG. 9B). Through PIV analysis, it was found that Rho-CA cells maintain approximately the same level of correlation and alignment with their neighboring cells as the WT cells during collective migration on corresponding ECM conditions (see e.g., FIG. 12B, FIG. 12C). Consistent with the idea that RhoA activity and cytoplasm-to-nucleus YAP translocation are both associated with ECM stiffness-dependent mechanotransduction, it was found that overall cytoplasmic localization of YAP in Rho-CA cells was lower than in WT cells (see e.g., FIG. 12D, FIG. 12E). After arriving in a stiff secondary region, the percentage of Rho-CA cells with the nuclear localization was approximately halved due to the preconditioning on a soft primary ECM compared to a stiff one (see e.g., FIG. 12D). A softer past ECM continues to hold back YAP translocation towards the nucleus even after the arrival of cells into a stiff ECM, which is consistent with observations for the WT cells. On a soft secondary region, Rho-CA cells preconditioned on a stiff ECM had ˜35% nuclear localization compared to none for those coming from a soft ECM (see e.g., FIG. 12D). Thus, the preconditioning on a stiff ECM enables Rho-CA cells to localize YAP within the nucleus even after they arrive in a soft secondary ECM.

### Example 2: Past Matrix Stiffness Primes Epithelial Cells and Regulates their Future Collective Migration Through Mechanical Memory

This example describes how past matrix stiffness primes epithelial cells and regulates their future collective migration through a mechanical memory.

During morphogenesis and cancer metastasis, grouped cells migrate through tissues of dissimilar stiffness. Although the influence of matrix stiffness on cellular mechanosensitivity and motility are well-recognized, it remains unknown whether these matrix-dependent cellular features persist after cells move to a new microenvironment. As disclosed herein, whether priming of epithelial cells by a given matrix stiffness influences their future collective migration on a different matrix—a property referred to as the ‘mechanical memory’ of migratory cells—is studied. To prime cells on a defined matrix and track their collective migration onto an adjoining secondary matrix of dissimilar stiffness, a modular polyacrylamide substrate was developed through step-by-step polymerization of different PA compositions. As disclosed herein, it is reported that epithelial cells primed on a stiff matrix migrate faster, display higher actomyosin expression, form larger focal adhesions, and retain nuclear YAP even after arriving onto a soft secondary matrix, as compared to their control behavior on a homogeneously soft matrix. Priming on a soft ECM causes a reverse effect. The depletion of YAP dramatically reduces this memory-dependent migration. The results present a previously unidentified regulation of mechanosensitive collective cell migration by past matrix stiffness, in which mechanical memory depends on YAP activity. The persistent influence of cellular mechanosensitivity on cell migration can be referred to as the ‘mechanical memory of migratory cells’. As described herein, it was discovered that collectively migrating cells remember their past matrix stiffness as they move across mechanically dissimilar microenvironments. This was discovered by developing a modular polyacrylamide (mPA) substrate comprising contiguous primary and secondary ECM regions of independently defined stiffness. It was discovered that a monolayer of cells primed on a stiff ECM migrated faster and in a more coordinated manner after arriving on a soft secondary ECM, as compared to those cultured on a homogeneously soft ECM. Nuclear translocation of YAP persisted even after cells arrived onto a softer secondary ECM and shRNA-mediated depletion of YAP dramatically blunted this mechanical memory-dependent cell migration. Taken together, the results as described herein bring an additional dimension to the existing framework of mechanosensitive migration of epithelial cells in response to their current microenvironment. Mechanical memory in migratory cells may have a particular significance to cancer metastasis, where future invasion potential of escaped cancer cells may be predicted by exploiting their persistent dependency on the primary tumor microenvironment stiffness.

Modular Polyacrylamide (mPA) Hydrogels

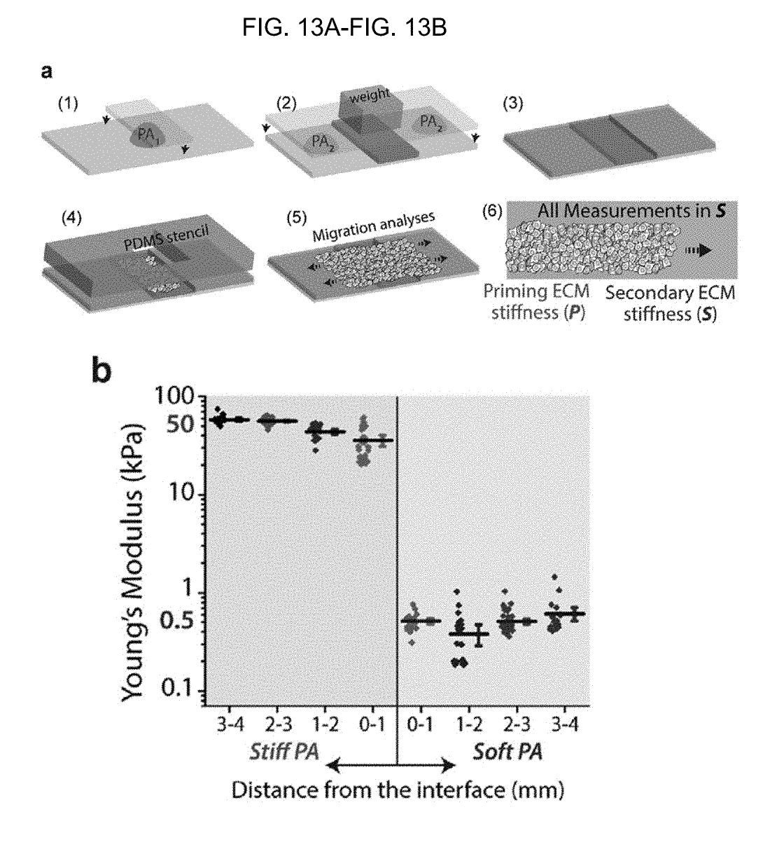

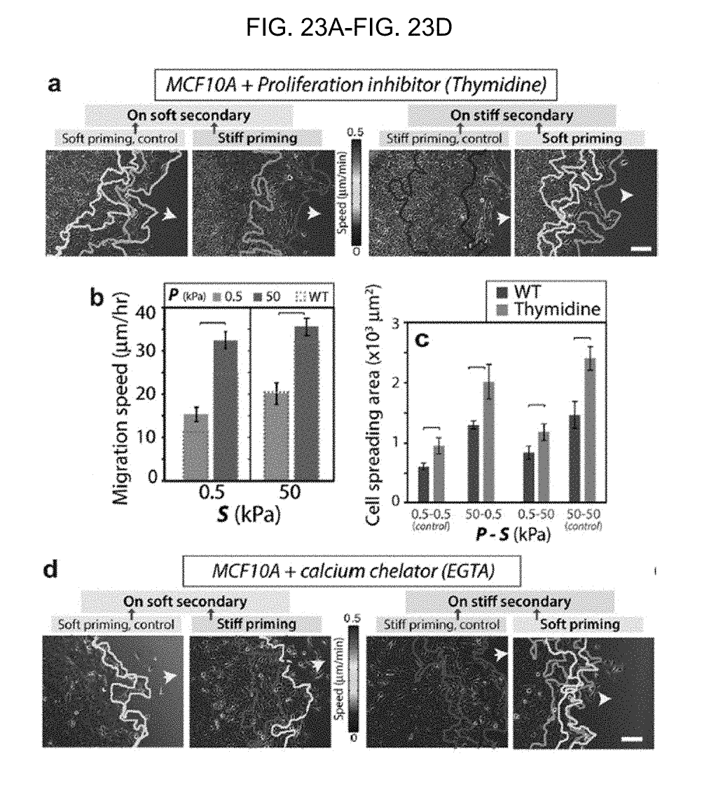

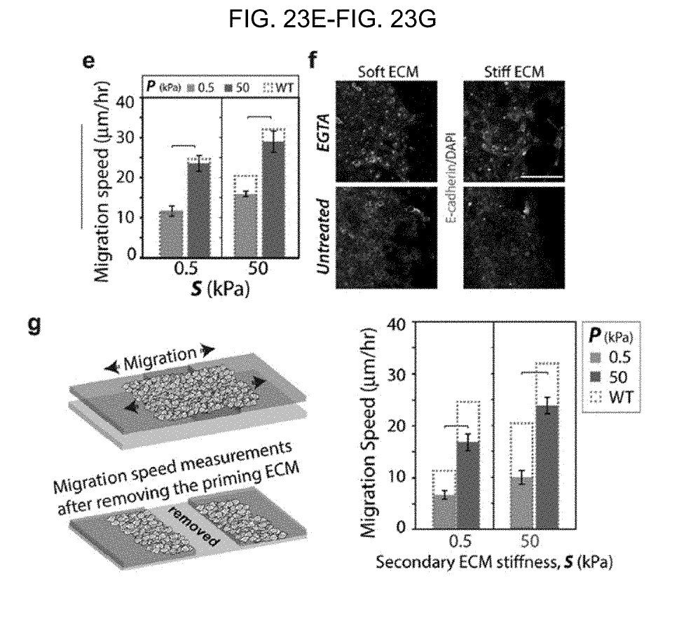

Contiguous polyacrylamide gels with distinct modules were fabricated through a step-by-step polymerization of PA solutions of defined compositions. Precursor solutions containing the acrylamide:bisacrylamide (A:B) with percentages of 4:0.2% or 12:0.6% were mixed with 0.5% Ammonium Persulphate (APS) and 0.05% Tetramethylethylenediamine (TEMED). Red fluorescent carboxylate-modified beads of 200-nm diameter were added at 0.1% concentration to the stiff PA precursor solution to identify the interface between dissimilar ECM modules. Next, a volume of PA precursor solution sufficient to achieve a gel thickness of 100 mm was dispensed on the coverslip and covered with a glass slide of defined size to confine the spreading of the PA droplet in each module of the substrate. This step was repeated for all three modules (see e.g., FIG. 13A) to fabricate the entire modular PA (mPA) hydrogel substrate. After polymerization, mPA gels were sterilized for 1 h under UV light. FIG. 31 is another illustration of an example of the method of preparing the tumor invasion device (see e.g., also FIG. 9, FIG. 13A, FIG. 23G). Subsequently, PA gels were treated with 0.5 mg/ml of sulfosuccinimidyl 6-(40-azido-20-nitrophenylamino) hexanoate (Sulfo-SANPAH) (Thermo Fisher Scientific) prepared in 50 mM HEPES buffer (Santa Cruz Biotechnologies) and crosslinked to the mPA surface upon activation with 365 nm UV for 10 min. After extensive washing with 50 mM HEPES buffer, mPA gels incubated with 0.05 mg/mL of rat-tail collagen type I (Santa Cruz Biotechnologies) overnight at 4° C.

Mechanical Characterization of PA Hydrogels

Atomic Force Microscopy (AFM) measurements of polyacrylamide gels were performed using an MFP-3D-BIO atomic force microscope (Asylum Research, Santa Barbara, Calif.). Olympus TR400PB AFM probes with an Au/Cr coated silicon nitride cantilever and pyramidal tip were used, with spring constants ranging from 20 pN/nm to 30 pN/nm, as measured by thermal calibration. Force maps in square regions of 40 mm edge length, with 169 points per force map, were taken at equal spacing across the gels. Measurements were performed across at least 4 mm length on each side of the interface dividing the primary and secondary ECM regions, as shown in FIG. 13B. Elastic moduli were extracted from force curves using a modified Hertz model.

Cell Culture and Collective Migration Assay

Human mammary epithelial non-tumorigenic MCF10A cells were cultured in DMEM-F12 (GE Healthcare Life Sciences), supplemented with 5% horse serum (Invitrogen), 20 ng/mL epidermal growth factor (EGF, Miltenyi Biotec Inc), 0.5 mg/mL hydrocortisone (Sigma-Aldrich), 100 ng/mL cholera toxin (Sigma-Aldrich), 10 μg/mL insulin (Sigma-Aldrich), and 1% (v/v) penicillin-streptomycin (Sigma-Aldrich). Tumorigenic mammary epithelial MCF7 cells were grown in DMEM (Sigma-Aldrich) containing 10% fetal bovine serum (FBS), 1% (v/v) penicillin-streptomycin (Sigma-Aldrich), and 1% non-essential amino acids (0.1 mM). Human epidermoid carcinoma A431 cells were grown in DMEM (Sigma-Aldrich) containing 10% fetal bovine serum (FBS), 1% (v/v) penicillin-streptomycin (Sigma-Aldrich), 1% sodium pyruvate (Sigma-Aldrich), 1% sodium bicarbonate (Sigma-Aldrich), and 1% non-essential amino acids (0.1 mM). A PDMS stencil was designed and fabricated with a rectangular opening in the center, restricting the culture of epithelial monolayer within the central module (primary ECM) of mPA substrate (see e.g., FIG. 13A). PDMS stencils were air-dried, cleaned with 70% ethanol, and sterilized under UV light for 2 h. Afterwards, stencils were passivated overnight, with a sterile solution of 1% Pluronic and 1% Bovine serum albumin (BSA) in Phosphate buffered saline (PBS) to avoid cell adhesion, and carefully positioned on the PA gels. Cell suspension with 2×10⁴ cells was dispensed into the PDMS reservoir covering the primary ECM region and left to grow for a prescribed duration of priming (1, 2, or 3 days) at 37° C. in a 5% CO₂ humidified incubator. After this incubation period, the PDMS stencil was lifted off to allow cell sheet to migrate into the adjoining secondary ECM. Additional media was added to each well.

For proliferation inhibition experiments, cells were treated with 2 mM thymidine (Sigma-Aldrich) after at least 6 h of cell seeding, to allow adequate attachment of cells to the substrate. For calcium chelation experiments, cells were incubated with 4 mM of EGTA (Sigma-Aldrich) after 3-day priming.

Live-Cell Imaging

Time-lapse microscopy was initiated 2 h after removing the PDMS and time-lapse imaging was carried out in the phase contrast on an inverted microscope (Zeiss Cell Observer) equipped with an incubator capable of maintaining an environment with 37° C. temperature and 5% CO₂ level. Images were acquired with a 10× objective. Two successive images of the same field were taken at 20 min time interval. Motion of the leading edge of the cell monolayer was manually tracked by recording the position of cell border using a homemade macro in ImageJ. Subsequently, coordinates of the leading edge were imported into a custom-made program in MATLAB and its advancement was calculated by averaging the distance of each point on the leading edge from the primary-secondary ECM interface. Leading edge speed was defined as the ratio of the leading edge advanced distance and the time course of migration. For single cell migration analysis, individual movies were imported to ImageJ software (National Institutes of Health), and single cell migration trajectories were extracted using the Manual Tracking plugin. Cell migration tracks were subsequently analyzed to obtain migration speed and velocity angle distribution. Migration speed was calculated as a ratio between the sum of distances traveled by the cell between each time point and the total time. For each time interval, the angle between instantaneous velocity vector and the x axis was calculated and plotted the angle distribution for the entire migration tracking period.

Particle Image Velocimetry (PIV) and Monolayer Dynamics

PIV analyses were performed to quantify spatiotemporal velocity distribution of velocity magnitudes by implementing the PIVlab package in MATLAB. To reduce the systematic biases in subpixel resolution, PIV was iteratively implemented for up to three passes of 64, 32, and 16 pixel windows with 50% overlap between adjacent windows. Displacement and velocity (displacement/time interval) vectors were calculated by comparing the displacement of a window between two successive images. The velocity field was expressed in mm/min. PIV analysis yielded two components of velocity at each point (i,j), namely lateral (uij, perpendicular to the direction of group migration) and axial (vij, along the direction of group migration). After obtaining the velocity vectors, cell alignment and cell-cell coordination were evaluated in terms of order parameter and correlation length [25]. The order parameter was defined as the cosine of the angle that the velocity vector makes with the principal direction of migration. This principle direction is the vector sum of all velocity vectors analyzed in a given frame. The order parameter varies from +1 (for velocity vectors parallel to the strip and directed along the direction of migration of the cell sheet) to −1 (for vectors that are directed opposite to the direction of migration of the cell front) through 0 (for vectors aligning perpendicular to the direction of the monolayer migration). Correlation lengths were calculated according to the method described earlier [25]. To demonstrate the time evolution of monolayer motion, kymographs of velocity magnitude and order parameter were plotted. After obtaining the velocity vectors for every pixel of the monolayer at a given time instant, kymographs were computed by averaging the velocity magnitude and order parameter of individual velocity vectors in x direction over the y coordinates in every time point for time period of 12 h after monolayer crossed the interface.

Immunofluorescence and Confocal Microscopy

After day 5, cells in the migration assay were rinsed with cold 1×PBS for 2-3 min and fixed in 4% Paraformaldehyde (PFA) at room temperature (RT) for 10 min. After washing again with PBS, cells were incubated with 1% bovine albumin serum (BSA) (EMD millipore) overnight at 4° C. Next, cells were washed with PBS for 30 min, and incubated in the primary antibody solution for yes-associated protein (YAP) (1:100; Santa Cruz) or phosphorylated myosin light chain (pMLC) (1:100; Cell Signaling Technology) prepared in 1% BSA, and stored overnight at 4° C. Samples were washed and incubated with appropriately matched secondary antibodies (Invitrogen) for 1 hour at RT. After thoroughly rinsing the substrates with PBS, DAPI (1:250; Santa Cruz Biotechnology) and Phalloidin (1:200; Invitrogen) was added for 30 min at RT. Finally, substrates were rinsed again with PBS and stored at 4° C. before imaging. Images were recorded at RT using a laser-scanning confocal microscope (Ziess LSM 730; Carl Ziess Microlmaging; Germany) at 20× or 40× objective, and confocal stacks were acquired at 1 mm interval. Image acquisition parameters including laser intensity and exposure times were maintained at the same level to ensure quantitative image analysis. Experiments were performed in triplicates, and the images used for analysis were randomly selected from 10 to 15 fields of view for each condition.

Quantitative Image Analysis

Captured z-stacks were imported to ImageJ (NIH) as LSM files, and the stacks were projected with the maximum intensity setting. To quantify the subcellular YAP activity, the mean fluorescence intensity of YAP was measured in the nucleus and the cytoplasm. Afterwards, the nuclear-to-cytoplasmic ratio of YAP intensity was computed and plotted to compare YAP localization across experimental conditions. At least 50 cells were randomly selected for analysis from 10 to 15 field of views selected from three independent experiments. To quantify pMLC intensity, the mean protein intensity per cell in a given region of interest (ROI) was calculated after subtracting the background signal (corresponding to the intensity of the negative control) from the total intensity in the z-stack (sum over all pixels of slices) and normalized to the number of cells in the corresponding region. The number of cells in a given ROI was obtained by manually counting cell nuclei in the maximum projected DAPI image. To compare between different experimental conditions, pMLC intensity per cell values were normalized to the values obtained for cells cultured on control stiff substrates. To analyze actin fiber orientation, the resulting z-projected image of phalloidin (sum over all pixels of slices) for each cell was analyzed using OrientationJ algorithm in ImageJ. For each cell, the degree of stress fiber alignment was calculated in terms of coherency within a defined region of interest through ImageJ, which varies between 0, indicating isotropic distribution, and 1, indicating highly aligned structures. Subsequently, the average fiber alignment was obtained by normalizing to the fiber alignment value obtained for cells cultured on stiff control substrates. The mean spreading areas of cells located at different distances with respect to the leading edge on various substrates were measured using ImageJ software by manually drawing the border of cell from phalloidin images and evaluating the resulting cell areas. At least 40 cells were analyzed from three independent experiments for each experimental condition. To measure the size of focal adhesions (FAs), confocal z-stack images of paxillin staining were acquired. FA area was computed by outlining punctate focal adhesions in binarized paxillin images and using the “Measure” tool in ImageJ to calculate FA area. At least 15 cells were analyzed for each experimental condition.

shRNA Knockdown

To deplete YAP, the lentiviral pFLRu vector containing either Scramble (shSCRM) or anti-YAP shRNA (shYAP), and puromycin resistance was used. HEK293T cells were cultured in DMEM (Gibco) supplemented with 10% heat-inactivated fetal bovine serum (FBS), 200 μM L-glutamine (Cellgro), and penicillin-streptomycin. HEK293T cells were transfected with lentiviral DNA using the TransiT LT1 transfection reagent per manufacturer protocol (Mirus). Virus was harvested from 293 T media 48 h after transfection and used to transduce MCF10A cells. Puromycin (Sigma-Aldrich) was used in cell selection and maintenance at a concentration of 1.5 μg/mL.

Western Blotting

Cells were lysed in RIPA buffer (50 mM Tris, pH 8.0, 150 mM NaCl, 1% NP-40, 0.5% sodium deoxycholate, 0.1% SDS) supplemented with 200 nM phenylmethylsulfonyl fluoride (PMSF), 2 μg/mL aprotinin/leupeptin, 2 mM pepstatin A, 1 mM Na3VO4, and 2 mM NaF. The lysates were cleared by centrifugation at 12,000 rpm at 4 C for 10 min, and the concentrations were determined by Bradford assay (Bio-Rad). Equal amounts of protein were boiled in SDS sample buffer for 10 min, resolved by SDS 10%-PAGE, and transferred to polyvinylidene difluoride (PVDF) membranes (Millipore) in transfer buffer (25 mM Tris, 192 mM glycine, 5% methanol). The membranes were blocked with TBST (25 mM Tris, pH 7.4, 150 mM NaCl, 2 mM KCl, 0.5% Tween 20) containing 5% skim milk powder or bovine serum albumin (BSA) and probed overnight with the indicated primary antibodies. Bound antibodies were detected by horseradish peroxidase (HRP)-conjugated secondary antibodies and developed with SuperSignal West Pico and/or West Femto enhanced chemiluminescence (ECL) (Pierce). Images were collected on a Bio-Rad ChemiDoc XRSρ and analyzed using ImageJ software (NIH). The following antibodies were used: rabbit anti-YAP/TAZ (Cell Signaling Technology, D24E4, mAb #8418; 1:1000), mouse anti-Actin (Sigma, A5316; 1:25,000).

### Statistical Analysis

Unless specified otherwise, results are reported as the mean±Standard Error (SE). To identify the significant differences between two experimental conditions, an F-test was performed to determine whether equal variance could be assumed. Next, Student's t-test was used to determine significant differences between two groups. All statistical analyses were performed using the Data Analysis toolbox in Microsoft Excel. Differences were considered to be significant for P<0.05.

Fabrication of Dual-Stiffness Contiguous Substrates

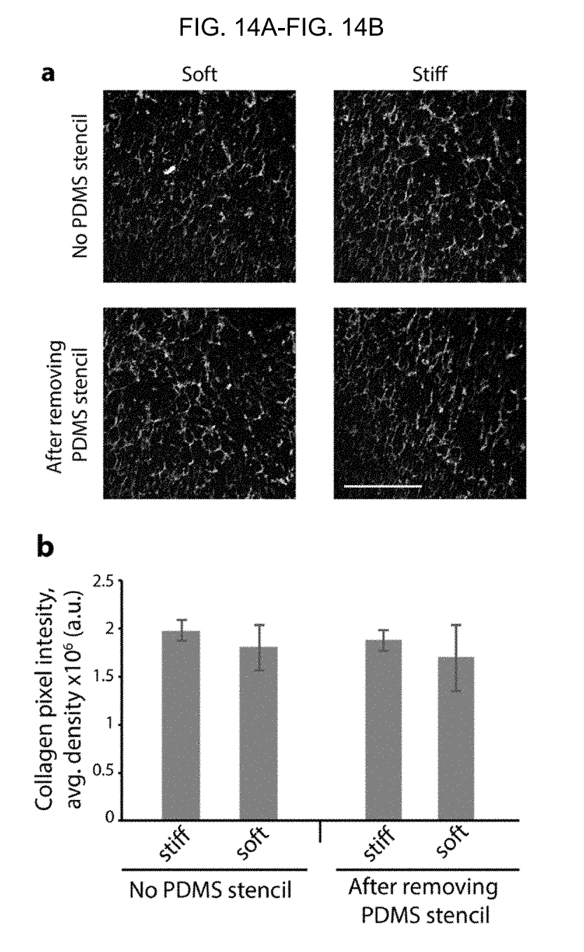

To prime epithelial cells on a given ECM and track their subsequent collective migration onto an adjoining ECM of dissimilar stiffness, a modular-PA (mPA) hydrogel substrate through step-by-step polymerization of two different PA compositions was fabricated [22] (see e.g., FIG. 13A). Through AFM-based mechanical characterization of these gels, as done previously [3, 23], it was found that the soft and stiff regions have corresponding Young's moduli of approximately 0.5 and 50 kPa, respectively (see e.g., FIG. 13B). These measurements also verified that the ECM stiffness in the primary and secondary ECM regions varied in a step-wise manner, as expected. These stiffness values are not chosen to match any specific tissue context; rather, this range is designed to explore the biophysical effects of cellular priming across a steep change in matrix stiffness. Next, a PDMS stencil was designed for culturing an epithelial monolayer restricted within the central section of the mPA gel, which is referred to as the ‘primary’ ECM. After priming the cell colony on the primary ECM for a prescribed duration, the PDMS stencil was removed to enable collective migration of the cell sheet into the surrounding ‘secondary’ ECM of independently defined stiffness. It was verified that the coating of collagen I on the PA gel did not vary with stiffness or the removal or PDMS stencil (see e.g., FIG. 14). In this system, epithelial cells seamlessly move across a contiguous substrate composed of mechanically distinct regions, whose stiffness can be tuned over two orders of magnitude. Thus, it was possible to study the effect of past ECM stiffness on future cell behavior without having to detach and re-culture cells on a new substrate. Comparison of cell behavior on a given secondary ECM with respect to varying priming ECM stiffness would reveal whether migratory epithelial cells store mechanical memory of their past mechano-regulated state.

Leading Edge Migration Depends on the Past ECM Stiffness

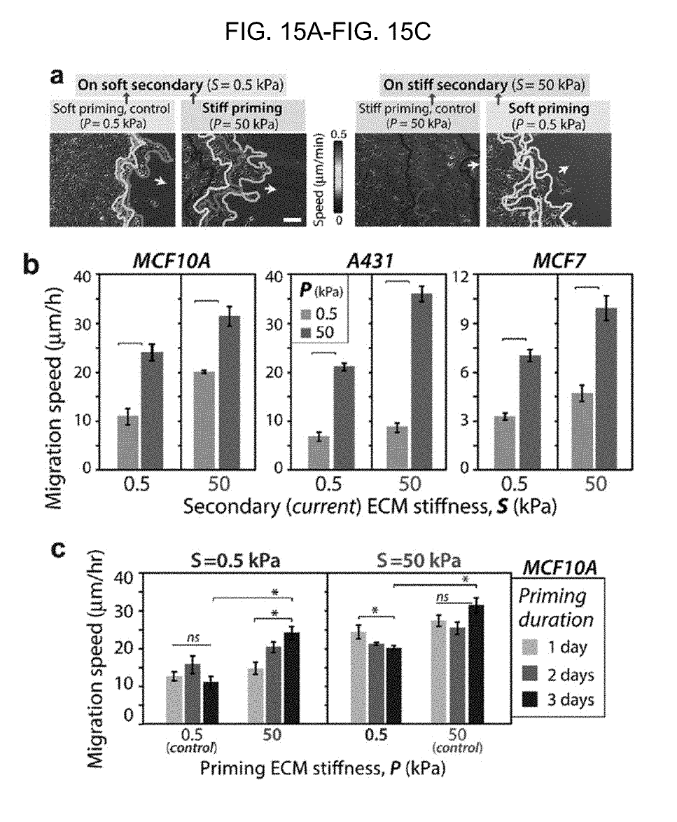

To assess the effect of past ECM stiffness on collective cell migration, MCF10A human mammary epithelial cells were cultured on primary ECM stiffness (P) for 3 days and then allowed the cells to migrate onto a secondary ECM (stiffness ‘S’). A time-lapse microscopy was performed for an additional 2 days, i.e., between days 4-5 from the time of initial culture, and manually traced the leading edge over time, as illustrated in FIG. 15A. On substrates with homogeneous stiffness (P=S), the average leading edge migration on stiff ECM (50 kPa) was 30 mm/h, which was three times higher than its value on the soft ECM (0.5 kPa) (see e.g., FIG. 15B). This stiffness-dependent collective cell migration speed is consistent with previous studies [6,7]. When the cell monolayer was first primed on a stiff primary ECM (P=50 kPa), the leading edge migration speed measured on the adjacent soft secondary ECM was ˜2.5 times higher than the control case of homogeneously soft ECM (see e.g., FIG. 15B). This enhanced leading edge migration could be attributed to the stiffness-dependent mechano-activated state of cells attained due to stiff priming, which was defined as the mechanical memory of collectively migrating cells. It was also found that the soft-primed cells migrated ˜40% slower than those primed on a stiff ECM (see e.g., FIG. 15B).

To test whether the duration of priming on the primary ECM influenced the memory-dependent migration, migration speed was measured after priming for 1 or 2 days. It was found that the influence of the primary ECM stiffness reduced with shorter priming duration (see e.g., FIG. 15C). Given that longer priming led to more pronounced mechanical memory-dependent collective migration, all results presented in this manuscript will correspond to a 3-day priming regimen, followed by 2 days of migration on the secondary ECM. All measurements are conducted on the secondary ECM, unless specified otherwise.

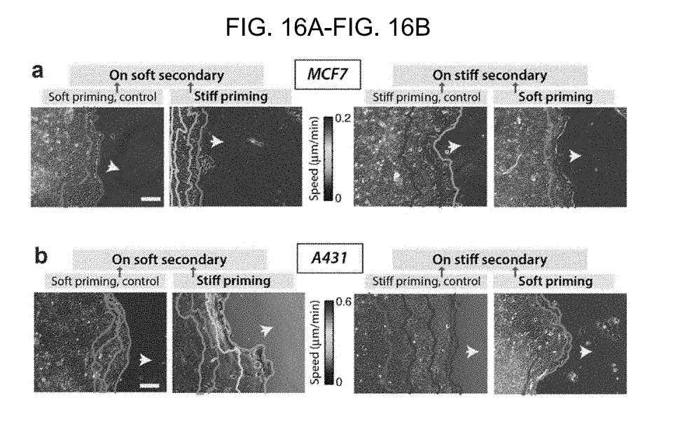

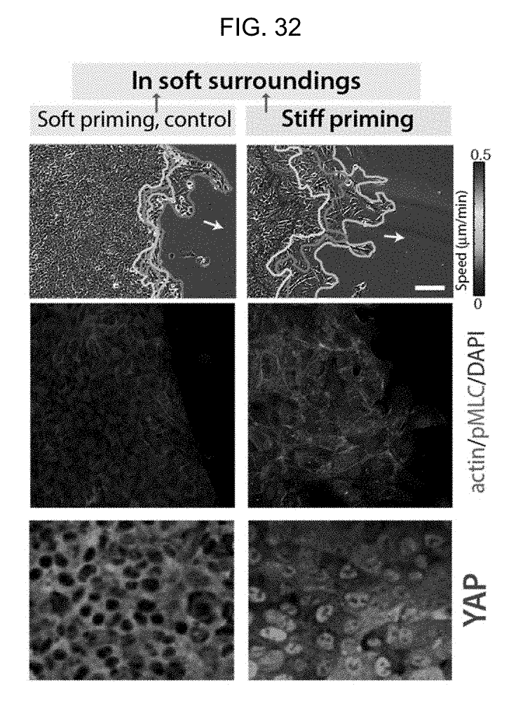

Leading-edge migration speed was also measured for MCF7 breast cancer cells and A431 human epidermoid carcinoma cells. While MCF7s were slower and A431s were faster compared to MCF10As, both of them exhibited robust mechanical memory-dependent migration (see e.g., FIG. 15B, FIG. 16). Staining identifying actin/pMPLC/DAPI and YAP provided additional evidence that mechanical memory has additionally been shown comparing cells that were primed in a soft environments versus a stiff environment (see e.g., FIG. 15A, FIG. 32).

Stiffer Primary ECM Predicts More Correlated Cell Migration

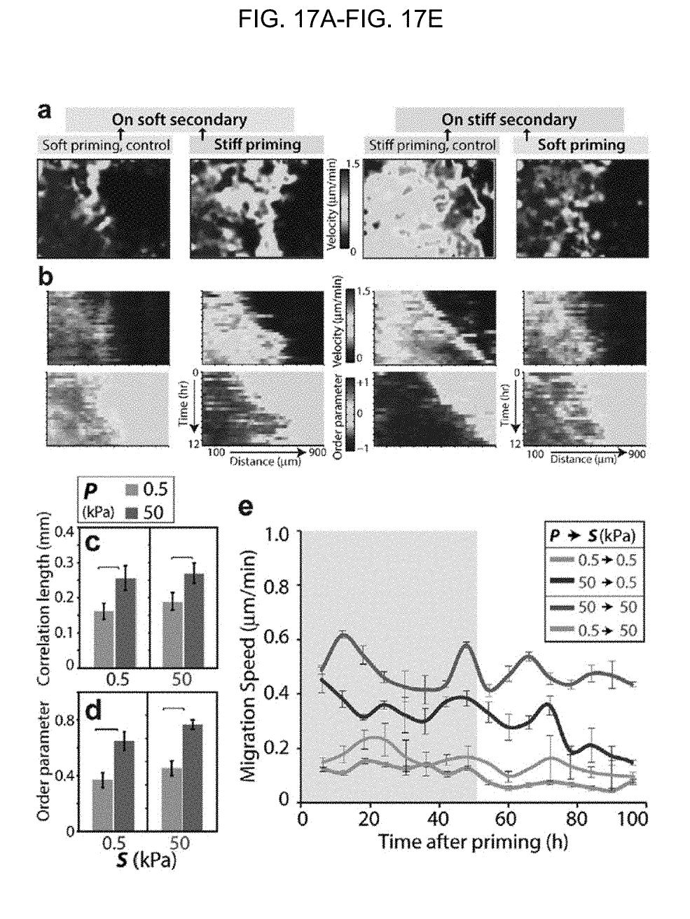

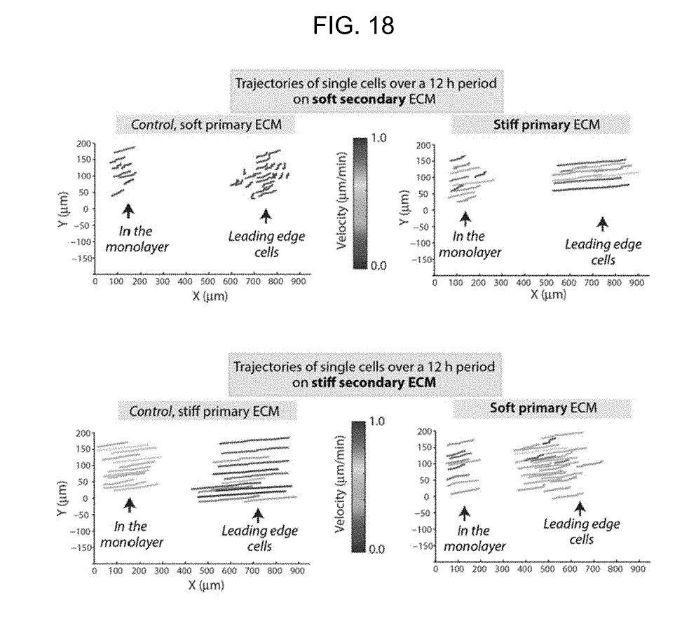

Using particle image velocimetry (PIV) analysis of phase contrast images of the migrating cell sheet over time [24], cellular motions within the epithelial monolayer were examined (see e.g., FIG. 17A). After arriving on a soft secondary ECM, stiff-priming led to high velocity magnitudes of the leading-edge cells, as compared to the soft primary ECM. Conversely, soft-priming significantly reduced the cell velocities. It was noted that cells behind the leading edge had lower velocity magnitudes in all cases. By plotting single cell trajectories over a 12 h period, it was confirmed that faster velocities of cells were at the leading edge compared to the ones within the monolayer (see e.g., FIG. 18).

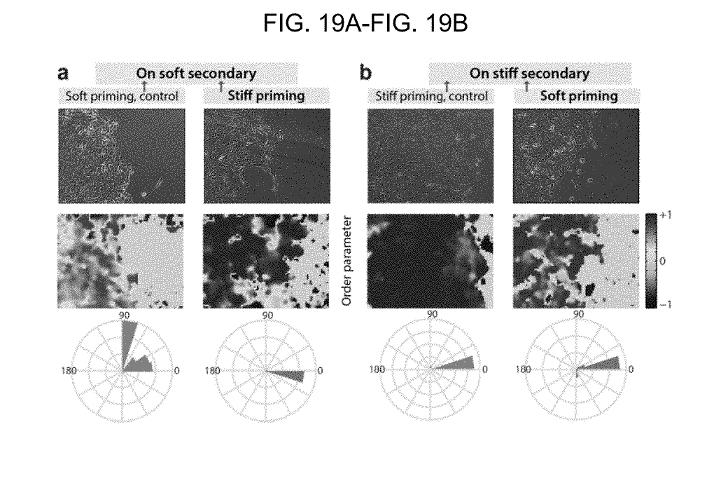

It was found that the correlation length, defined as the distance of correlation among velocity vectors of neighboring cells [25], for cells migrating on a homogeneously stiff ECM was ˜0.25 mm, which is ˜25% higher than its value measured on a purely soft ECM (see e.g., FIG. 17C). Thus, higher ECM stiffness enables larger portions of the cell sheet to migrate in a coordinated fashion, which is consistent with previous findings [6]. The stiff-primed cells migrated on a soft secondary ECM with ˜0.25 mm correlation length, the same as the value measured on a homogeneously stiff ECM. In comparison, soft-primed cells migrated with a lower correlation length of ˜0.2 mm on a stiff secondary ECM. The order parameter of the collective migration, defined as the angle between the velocity vectors and the direction of leading edge migration, on a homogeneously stiff ECM was more than twice its value on a soft ECM (see e.g., FIG. 17D, FIG. 19), indicating a more ordered collective migration on stiffer ECM [6]. The order parameter of stiff-primed cells remained high (˜0.6) on the soft secondary ECM. To assess the temporal progression of collective cell motility onto the secondary ECM, four representative videos of collective cell migration for each condition was selected and plotted how a column of cells traversed over a 12 h period (see e.g., FIG. 17B). Cells maintained their velocity magnitudes and order parameter over time, i.e., pixel intensities rarely varied over this 12 h period and distance (see e.g., FIG. 17B).

To further expand the temporal variation of migration speed across numerous samples, leading-edge migration speed was averaged at given time points and plotted these values over 4 days of migration after the 3-day priming (see e.g., FIG. 17E). It was found that the stiff-primed monolayers maintained at least ˜2 times higher speed on a soft secondary ECM (compared to purely soft ECM) for at least 3 days. This memory-based advantage in the migration speed of stiff-primed cells started to subside afterwards (see e.g., FIG. 17E). Thus, the presented effects of mechanical memory correspond to a phenotype maintenance within a temporal boundary, which is measured here as 3 days of collective migration in the secondary ECM.

Higher Actin/pMLC Expression and Adhesions Due to Stiffer Priming

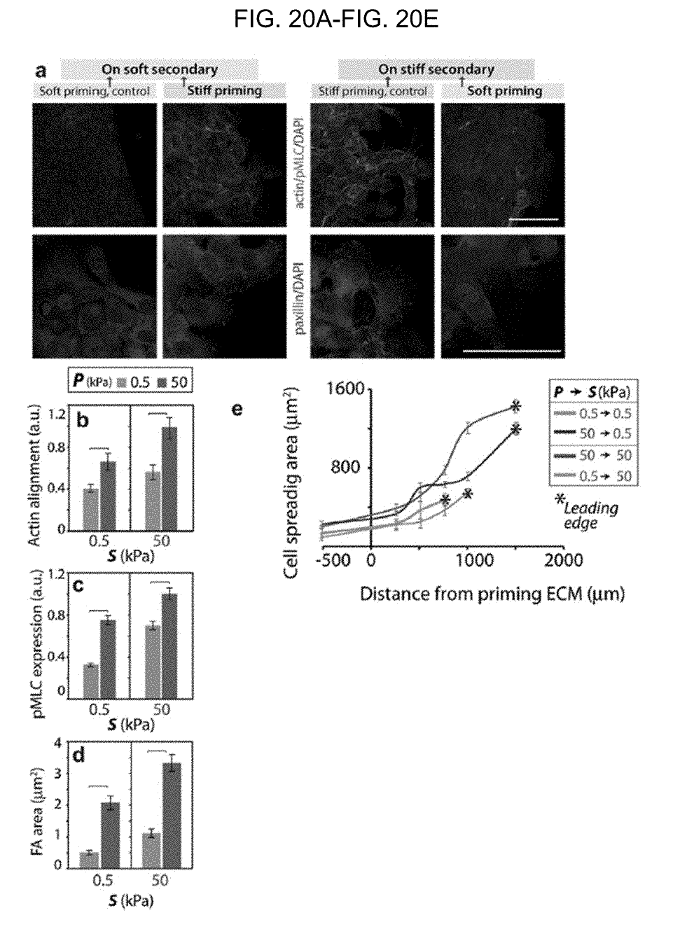

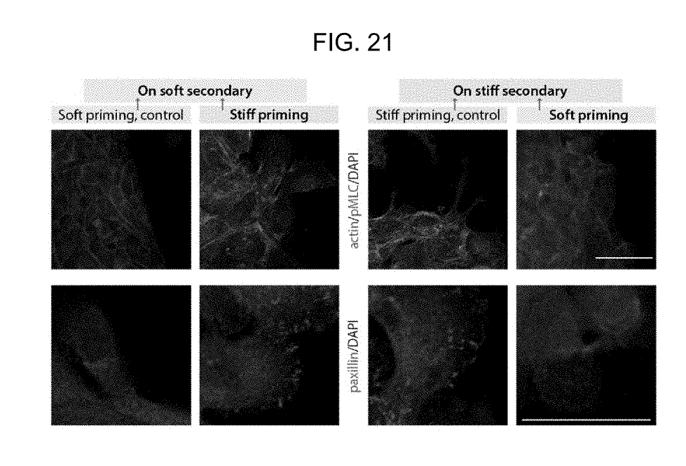

Because cell-ECM adhesions and actomyosin machinery are crucial for generating forces and driving cell motility [3, 6, 26], it was examined whether their subcellular expressions within individual cells near the leading-edge of the monolayer depended on the priming stiffness. To this end, F-actin and phosphorylated myosin light chain (pMLC) was stained and imaged in cells after their migration across the secondary ECM. It was found that actin fiber alignment (from phalloidin images), pMLC expression, and number of focal adhesion (FAs) (paxillin images) in cells on homogeneously stiff ECM were significantly higher than their average values measured on a soft ECM (see e.g., FIG. 20, FIG. 21). After stiff-priming, cells on a soft ECM exhibited larger FAs (˜4 times), pMLC expression (˜2.5 times) and actin alignment (˜1.6 times) compared to the control case of homogeneously soft ECM (see e.g., FIG. 20B-FIG. 20D). These results indicated that stiff-priming allowed the cells to maintain enhanced actomyosin machinery even after their traversal onto a soft secondary ECM. Conversely, the actin alignment and pMLC expression were reduced significantly after soft-priming.

To examine whether stiffness-sensitive cell spreading in the primary ECM could influence the leading-edge migration, cell areas were calculated across the monolayer. Notably, in all four matrix conditions, cell spreading reduced with distance from the leading edge, which led to a stiffness-insensitive spreading in the rear part of the monolayer (see e.g., FIG. 20E) and priming-dependent spreading near the leading-edge. Thus, cell spreading in the primary region cannot influence the leading-edge migration computed in the secondary region.

Alternate Hypotheses for Memory-Dependent Migration Due to Cell Proliferation and Signal Transmission

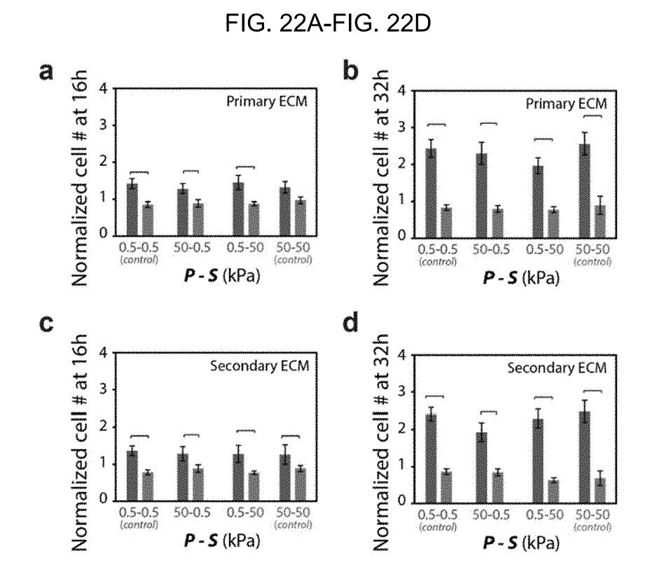

Although the results in FIG. 13, FIG. 15, FIG. 17, and FIG. 20 clearly show the influence of past ECM stiffness on cellular features in the secondary ECM, several alternative hypotheses other than the proposed ‘memory-storing’ abilities of migratory epithelial cells were examined that can explain the observed behavior. First, it was asked whether mechano-sensitive cell proliferation due to the stiffer primary ECM in the rear of the monolayer could influence leading edge migration. After inhibiting cell proliferation (thymidine treatment; FIG. 22), cell migration speeds increased due to increased spreading, yet the trend of mechanical memory-dependent migration held true (see e.g., FIG. 23A-FIG. 23B). In some conditions, higher migration speed after proliferation inhibition was observed, which may be attributed to increased cell spreading (see e.g., FIG. 23C).

Second, to attenuate inter-cellular force transmission, migration measurements in the presence of a calcium chelator (4 mM EGTA) were repeated, as used previously for this purpose [27, 28], which disrupts cell-cell communication (as shown through E-cadherin images in FIG. 23F). As a result, some cells break away from the monolayer. Overall, it was found that the memory-dependent migration of the leading edge was preserved despite the loss of cell-cell communication (see e.g., FIG. 23D-FIG. 23E).

Finally, to eliminate any possible communication between the primary and secondary regions, the entire primary region was physically removed along with the attached cells before measuring migration in the secondary ECM (1 day after complete priming). It was found that the memory-dependent migration persisted in this system (see e.g., FIG. 23G-FIG. 23H). These results, along with the ones presented above, confirm that the memory-dependent migration observed in the secondary ECM is independent of a direct communication with the primary ECM. Instead, the priming-dependent signals are likely stored within the cells and continue to dictate collective migration in the future.

Migratory Cells Store Mechanical Memory of Past ECM Stiffness Through YAP Activity

Described here and in the previous examples, the invasion-promoting signals continue to stay activated even after the cells arrive to a soft matrix. It was shown that this only happens when the cells were first primed on a stiff matrix.

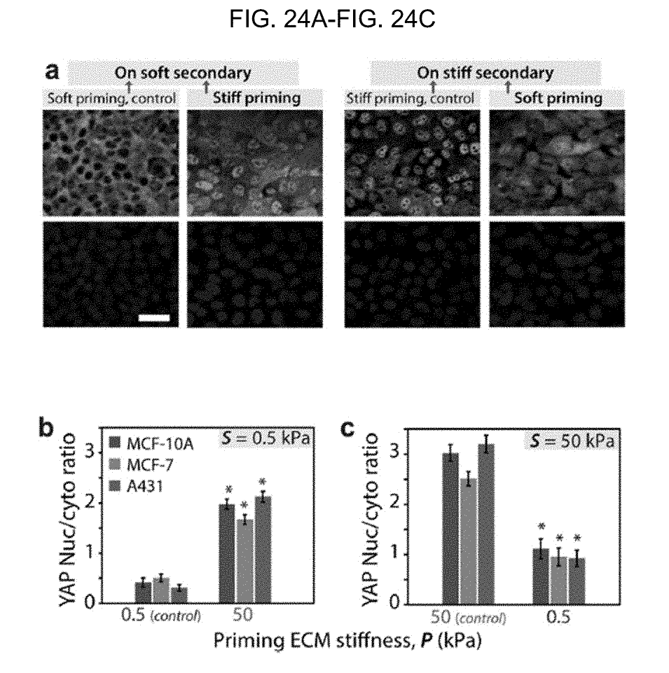

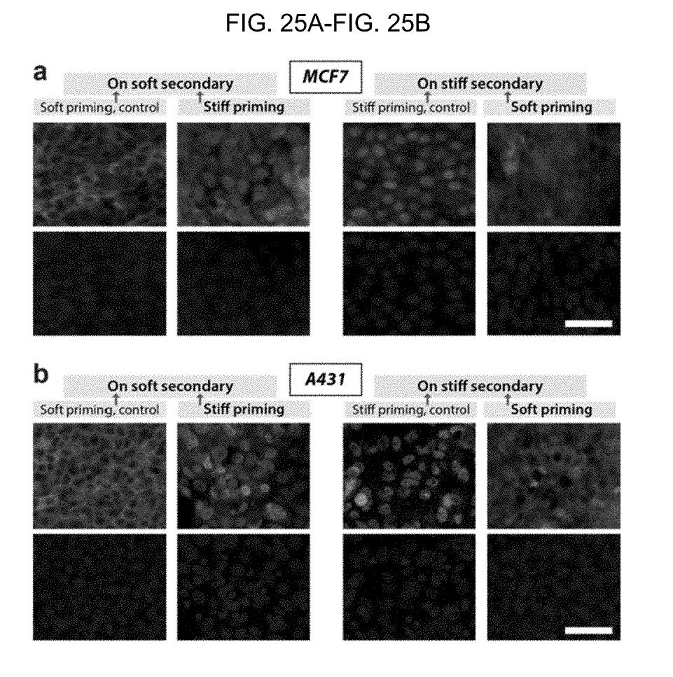

The observed memory-dependent collective cell migration indicates that the priming-dependent mechano-regulated state of cells may persist onto the new ECM. Given that nuclear translocation of YAP has been identified as a sensor of ECM stiffness [17, 29, 30], YAP subcellular localization was determined within MCF10A cells as they migrated across ECMs of dissimilar stiffness. The nuclear-to-cytoplasmic ratio of YAP fluorescent intensity was quantified in at least 40 cells from multiple fields. Consistent with previous findings (19), YAP expression was predominantly nuclear on homogeneously stiff ECM and cytoplasmic on homogeneously soft ECM (see e.g., FIG. 24). However, nuclear YAP localization of stiff-primed cells when measured in soft secondary ECM was more than four times its value on homogeneously soft ECM (see e.g., FIG. 24B). Thus, nuclear accumulation of YAP due to the stiffer past ECM persisted even after the cells migrated onto the adjoining softer region. Conversely, soft-primed cells on stiff secondary ECM showed less than ⅓rd nuclear YAP, compared to the homogeneously stiff ECM (see e.g., FIG. 24C). These measurements were repeated in MCF7 and A431 cells (see e.g., FIG. 24B-FIG. 24C, FIG. 25) and found that both of these cancer cell lines followed a similar dependence of subcellular YAP localization on the past matrix stiffness. Thus, cellular mechano-sensation of ECM stiffness through subcellular YAP localization could be a key mechanism for storing mechanical memory in migratory cells.

Inhibition of Mechanical Memory-Based Cell Migration Through YAP Depletion

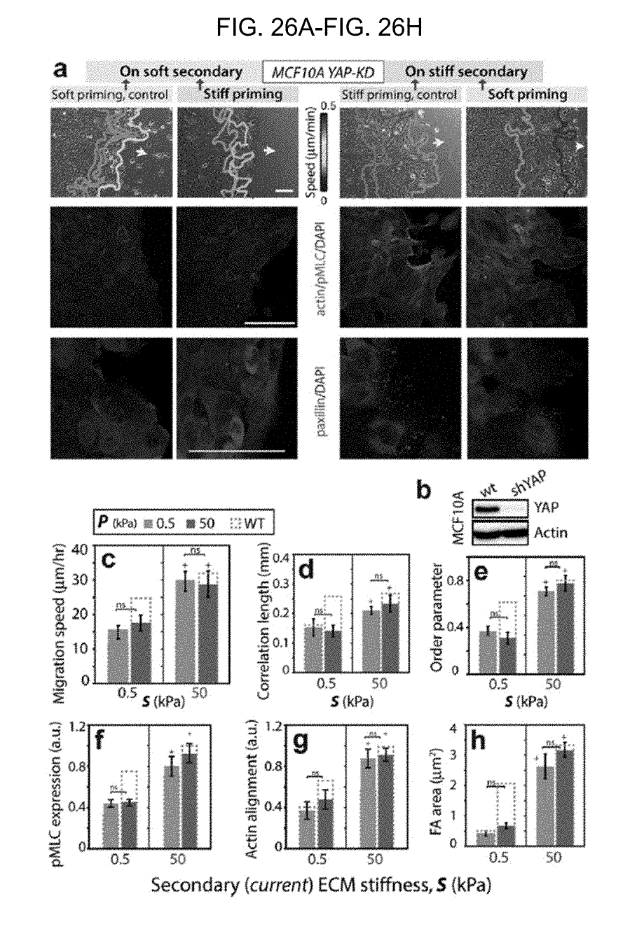

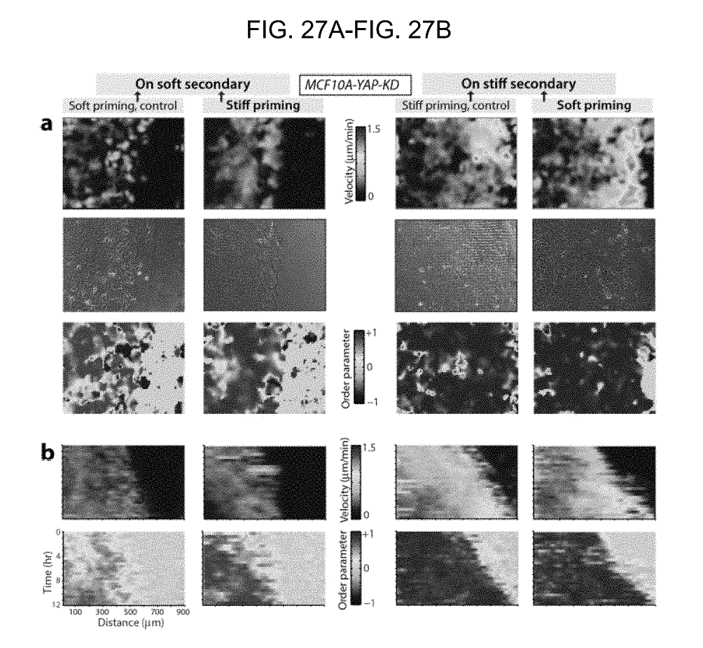

To further examine whether YAP activity is a requirement for memory-dependent migration, shRNA-mediated depletion of YAP in MCF10A cells (YAP-KD) was performed (see e.g., FIG. 26B), as described previously [18, 31]. It was found that the leading-edge migration speed of stiff-primed YAP-KD cells was ˜15 mm/h on a soft secondary ECM, which was similar to the control case of homogeneously soft ECM (see e.g., FIG. 26A, FIG. 26C). Even after priming on a soft ECM, YAP-KD cells migrated fast (˜30 mm/h) on a stiff secondary ECM, matching with the control case of a homogeneously stiff ECM. Thus, after YAP depletion, cells were unable to exploit prior priming. Notably, the YAP-depleted cells on homogeneously stiff ECM were almost twice as fast compared to those on homogeneously soft ECM (see e.g., FIG. 26A, FIG. 26C). Through PIV analysis of cellular motions within the monolayer, it was also found that the cells within the monolayer migrated in a memory-independent manner, with greater correlation and in a more ordered fashion on a stiffer ECM regardless of the primary ECM stiffness (see e.g., FIG. 26D-FIG. 26E, FIG. 27).

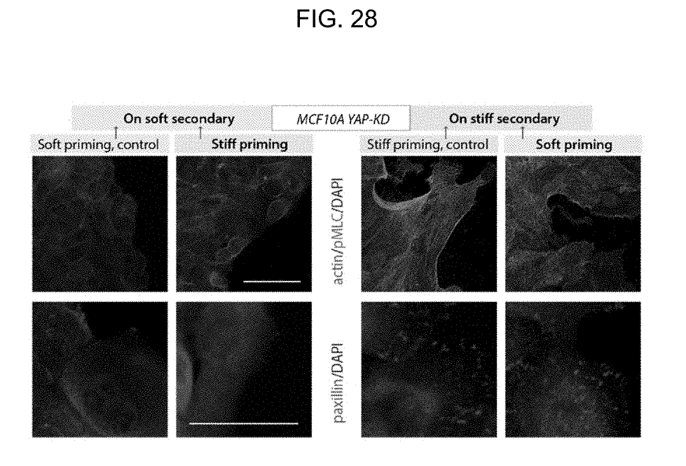

Given that YAP is a classic mechano-sensor [17], the observed ECM stiffness-dependent migration of YAP-depleted cells was unexpected. To understand the potential mechanism through which these YAP-KD cells continue to sense their immediate matrix stiffness, focal adhesions were imaged because they directly connect the cells to the matrix. Indeed, it was found that the average FA size of YAP-KD cells on stiff secondary ECM was more than 5 times higher compared to the values on the soft secondary ECM, regardless of primary ECM stiffness (see e.g., FIG. 26A, FIG. 26H). Similar mechanosensitive but memory-independent trends for actin fiber alignment, and pMLC expression was also found (see e.g., FIG. 26A, FIG. 26F, FIG. 26G, FIG. 28). These results demonstrate that YAP-KD cells are unable store a mechanical memory of past stiffness due to hampered YAP activity, but continue to sense the immediate matrix stiffness through focal adhesions.

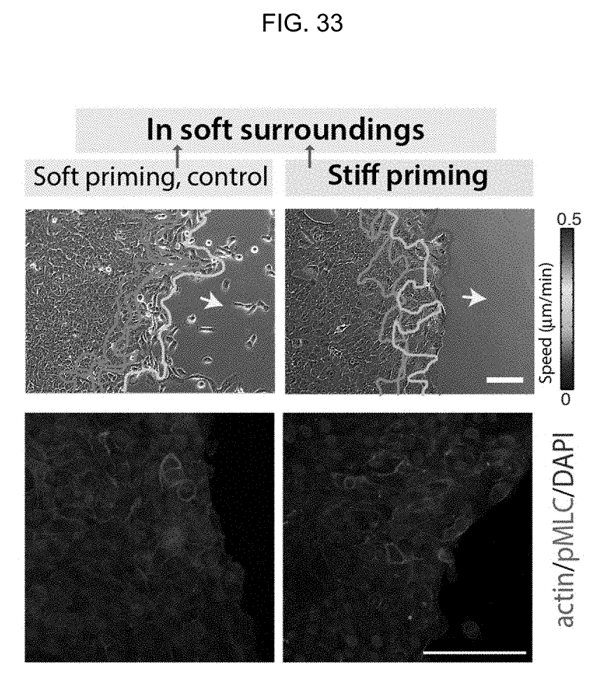

Here, YAP, a mechanical memory-specific target has been identified (see e.g., FIG. 33 for YAP-depleted breast epithelial cells). Cells were shown not to store mechanical memory after YAP-depletion. The cells were shown to migrate slowly after arriving in a soft environment, despite prior stiff-priming. Importantly, the YAP-depleted cells continue to show healthy signatures. Unlike other targets that destroy cellular structure to slow invasion.

Collagen as a Measure of Tumor Invasion

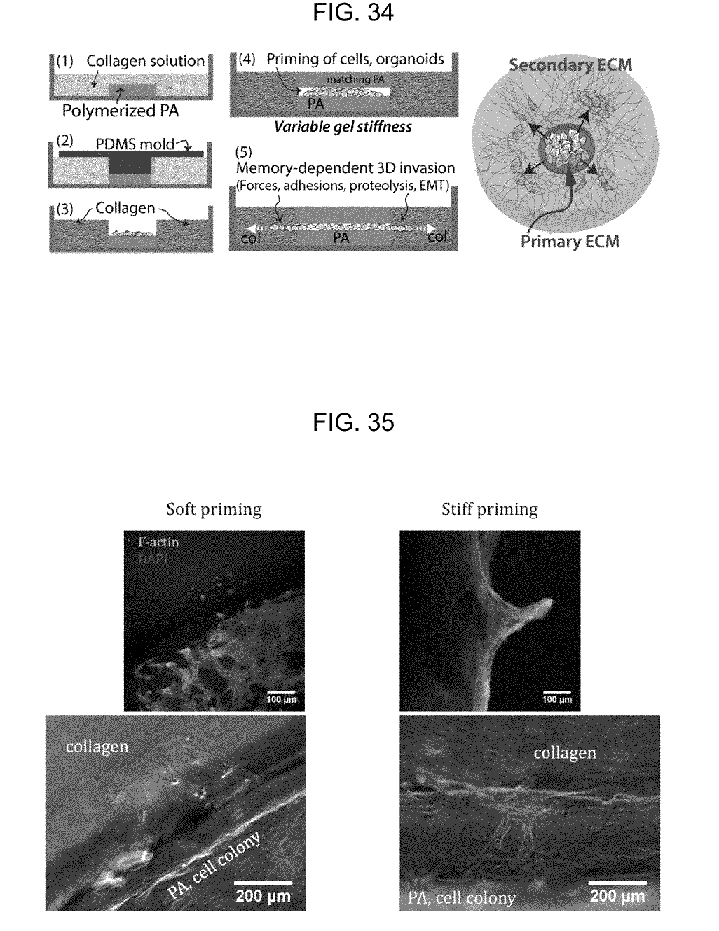

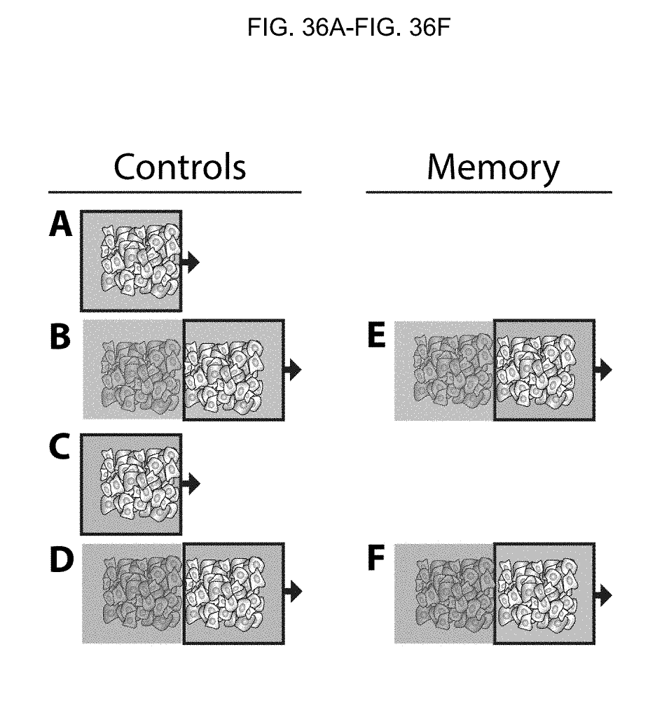

3D breast tumor invasion due to mechanical memory is modeled in the illustration of the device and illustrated in FIG. 34. More aggressive invasion of primary mouse breast tumor organoids (containing circulating tumor cells and cancer associated fibroblasts) and collagen deformation due to stiff priming was demonstrated (see e.g., FIG. 35).

### DISCUSSION

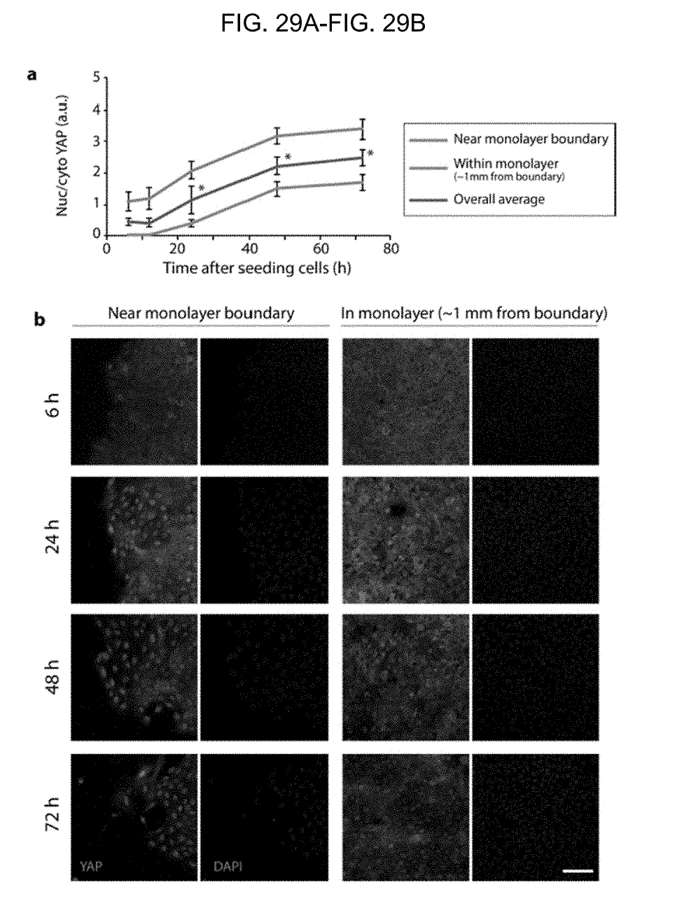

Plasticity in motile cells is manifested by variable modes of migration depending on the surrounding microenvironment [32]. In particular, cancer cells are uniquely equipped to exploit their plasticity to drive tumor invasion through distinct tissues. It has recently been identified that human mesenchymal stem cells store memory of their past exposure to matrix stiffness [19]. However, in migratory cells, it has remained a mystery whether their mechanics-regulated state persists even after they move to a new environment. If the mechanical properties of the tumor microenvironment are found to mechanically “train” the escaping cells, impacting their future ability to metastasize, this could be a critical missing piece of the puzzling unpredictability of cancer adaptation. To address this important gap in the understanding of cancer adaptation, it was asked whether collectively migrating cells retain a mechanical memory of their past ECM stiffness. Through measurements of collective cell migration across dual-stiffness substrates, it was shown that priming of an epithelial cell colony on a stiff ECM enhances its future collective migration even on a soft ECM. It was also shown that the enhanced migration of stiff-primed cells on soft ECM is not caused by the mechanosensitive differences in cell spreading, proliferation, or other mechanical signaling transmitted from the back of the monolayer. Indeed, when the cell colony was only primed for one day, the stiff primary ECM in the back was not able to enhance migration of cells on the soft secondary ECM. Thus, the memory-dependent cell migration is orchestrated by preserving the priming-enabled mechano-activated state of the cells onto the secondary ECM. It was discovered that less than 2 days of priming of cells on the primary ECM showed a substantially reduced mechanical memory-based migration. It is likely that the cells require 2-3 days to respond to matrix stiffness and accordingly localize YAP (nuclear or cytoplasmic). Indeed, the measurements of nuclear YAP localization within cells of a monolayer cultured on the stiff PA gel (50 kPa) at different time points reveal that YAP activation continues to rise over 3 days (see e.g., FIG. 29). Furthermore, this time-sensitive storage of YAP-dependent memory within the cells might require a transcriptional program, which is consistent with previous studies on mechanical-memory dependent responses of stem cells [19].

Given that YAP is a classic transducer of ECM [17] and its known role in storing memory in stem cells [19], its ability to retain information of past ECM stiffness was measured in collectively migrating cells. It was found that the stiffer priming predicted higher nuclear YAP localization—a sign of persistent mechano-regulated YAP activity. After YAP depletion, cell migration did not depend on past matrix stiffness, i.e., the mechanical memory was significantly diminished in these cells. According to this data, the YAP-depletion blunts mechanical memory without a significant loss of cellular mechanosensitivity to the immediate matrix stiffness.

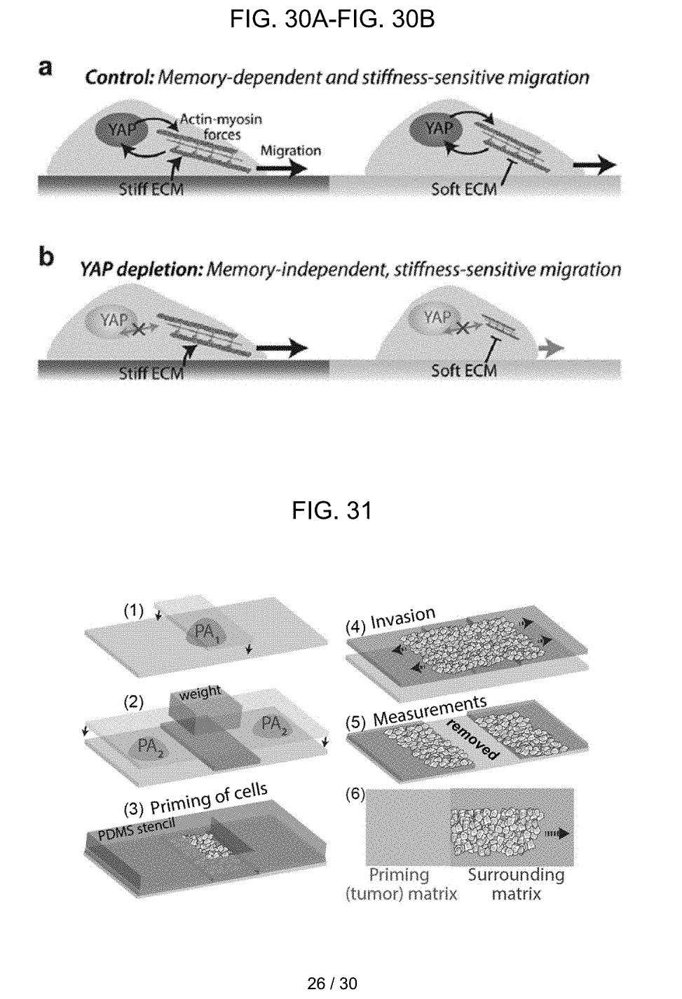

Our results point to a conceptual framework of mechanical memory-dependent cell migration in which migration-related cellular forces may be independently influenced by two factors (see e.g., FIG. 30). First, the priming-dependent YAP activity directly regulates actomyosin forces and migration (results from FIG. 20, FIG. 24). In cancer associated fibroblasts (CAFs), YAP activation has been shown to enhance and maintain a positive feedback loop with actomyosin contractility [4]. Similarly, fluid shear-dependent YAP activation has been shown to enhance protrusions required for migration [33]. Second, cells are able to sense immediate matrix stiffness through adhesions, despite YAP depletion (results from FIG. 26). Previously, it was shown that YAP-depleted CAFs maintain the mechanosensitive SNAIL1 protein level [18], which indicates that the cells are able to adopt alternate YAP-independent pathways for sensing matrix stiffness.

In summary, the present findings of the mechanical memory in migratory cells expand the basic understanding of cellular mechanotransduction, beyond the current framework of studying cell migration in the context of only the immediate microenvironment. The insight that stiffness of the past ECM can influence future collective migration opens the door to new hypotheses for a wide array of biological processes, wherever microenvironment-dependent cell motility plays a role, such as morphogenesis, wound healing, and cancer cell invasion. The knowledge of memory-storing abilities of invasive cancer cells and associated signaling targets can open new avenues for therapeutics and predictive modeling by exploiting their dependency on the primary tumor microenvironment and tuning their ability to adapt to foreign tissue environments. For example, the devices used for the experiments herein can be used as a device to test therapeutics or a tumor invasion model on a chip wherein the device can be used for finding novel metastasis targets through mechanical memory of cancer cells.

Having described several embodiments, it will be recognized by those skilled in the art that various modifications, alternative constructions, and equivalents may be used without departing from the spirit of the invention. Additionally, a number of well-known processes and elements have not been described in order to avoid unnecessarily obscuring the present invention. Accordingly, the above description should not be taken as limiting the scope of the invention.

Those skilled in the art will appreciate that the presently disclosed embodiments teach by way of example and not by limitation. Therefore, the matter contained in the above description or shown in the accompanying drawings should be interpreted as illustrative and not in a limiting sense. The following claims are intended to cover all generic and specific features described herein, as well as all statements of the scope of the present method and system, which, as a matter of language, might be said to fall therebetween.

### REFERENCES

[1] D. E. Discher, P. Janmey, Y.-I. Wang, Tissue cells feel and respond to the stiffness of their substrate, Science 310 (5751) (2005) 1139-1143.
[2] S. C. Wei, L. Fattet, J. H. Tsai, Y. Guo, V. H. Pai, H. E. Majeski, A. C. Chen, R. L. Sah, S. S. Taylor, A. J. Engler, J. Yang, Matrix stiffness drives epithelial-mesenchymal transition and tumour metastasis through a TWIST1-G3BP2 mechanotransductionpathway, Nat. Cell Biol. (2015).
[3] A. Pathak, S. Kumar, Independent regulation of tumor cell migration by matrixstiffness and confinement, Proc. Natl. Acad. Sci. U.S.A. 109 (26) (2012) 10334-10339.
[4] F. Calvo, N. Ege, A. Grande-Garcia, S. Hooper, R. P. Jenkins, S. I. Chaudhry, K. Harrington, P. Williamson, E. Moeendarbary, G. Charras, E. Sahai, Mechanotransduction and YAP-dependent matrix remodelling is required forthe generation and maintenance of cancer-associated fibroblasts, Nat. CellBiol. 15 (6) (2013) 637-646.
[5] A. Pathak, S. Kumar, Transforming potential and matrix stiffness co-regulate confinement sensitivity of tumor cell migration, Integr. Biol. 5 (8) (2013) 1067-1075.
[6] M. R. Ng, A. Besser, G. Danuser, J. S. Brugge, Substrate stiffness regulates cadherin-dependent collective migration through myosin-II contractility, J. Cell Biol. 199 (3) (2012) 545-563.
[7] R. Sunyer, V. Conte, J. Escribano, A. Elosegui-Artola, A. Labernadie, L. Valon, D. Navajas, J. M. Garcia-Aznar, J. J. Munoz, P. Roca-Cusachs, X. Trepat, Collective cell durotaxis emerges from long-range intercellular force transmission, Science 353 (6304) (2016) 1157.
[8] P. Friedl, D. Gilmour, Collective cell migration in morphogenesis, regenerationand cancer, Nature reviews, Mol. Cell Biol. 10 (7) (2009) 445-457.
[9] V. D. Varner, C. M. Nelson, Cellular and physical mechanisms of branching morphogenesis, Development 141 (14) (2014) 2750-2759.
[10] A. J. Ewald, A. Brenot, M. Duong, B. S. Chan, Z. Werb, Collective epithelial migration and cell rearrangements drive mammary branching morphogenesis, Dev. Cell 14 (4) (2008) 570-581.
[11] S. Nasrollahi, A. Pathak, Topographic confinement of epithelial clusters induces epithelial-to-mesenchymal transition in compliant matrices, Sci. Rep. 6(2016) 18831.
[12] P. Friedl, S. Alexander, Cancer invasion and the microenvironment: plasticity and reciprocity, Cell 147 (5) (2011) 992-1009.
[13] L. E. Dow, J. S. Kauffman, J. Caddy, K. Zarbalis, A. S. Peterson, S. M. Jane, S. M. Russell, P. O. Humbert, The tumour-suppressor Scribble dictates cell polarity during directed epithelial migration: regulation of Rho GTPase recruitment to the leading edge, Oncogene 26 (16) (2007) 2272-2282.
[14] A. Nicolas, B. Geiger, S. A. Safran, Cell mechanosensitivity controls the anisotropy of focal adhesions, Proc. Natl. Acad. Sci. U.S.A. 101 (34) (2004) 12520-12525.
[15] M. Chrzanowska-Wodnicka, K. Burridge, Rho-stimulated contractility drives the formation of stress fibers and focal adhesions, J. Cell Biol. 133 (6) (1996) 1403-1415.
[16] M. Reffay, M. C. Parrini, O. Cochet-Escartin, B. Ladoux, A. Buguin, S. Coscoy, F. Amblard, J. Camonis, P. Silberzan, Interplay of RhoA and mechanical forces in collective cell migration driven by leader cells, Nat. Cell Biol. 16 (3) (2014) 217-223.
[17] S. Dupont, L. Morsut, M. Aragona, E. Enzo, S. Giulitti, M. Cordenonsi, F. Zanconato, J. Le Digabel, M. Forcato, S. Bicciato, N. Elvassore, S. Piccolo, Role of YAP/TAZ in mechanotransduction, Nature 474 (7350) (2011) 179-183.
[18] K. Zhang, W. R. Grither, S. Van Hove, H. Biswas, S. M. Ponik, K. W. Eliceiri, P. J. Keely, G. D. Longmore, Mechanical signals regulate and activate SNAIL1 protein to control the fibrogenic response of cancer-associated fibroblasts, J. Cell Sci. 129 (10) (2016) 1989-2002.
[19] C. Yang, M. W. Tibbitt, L. Basta, K. S. Anseth, Mechanical memory and dosing influence stem cell fate, Nat. Mater. 13 (6) (2014) 645-652.
[20] C.-M. Lo, H.-B. Wang, M. Dembo, Y.-I. Wang, Cell movement is guided by the rigidity of the substrate, Biophysical J. 79 (1) (2000) 144-152.
[21] M. Raab, J. Swift, P. C. Dingal, P. Shah, J. W. Shin, D. E. Discher, Crawling from soft to stiff matrix polarizes the cytoskeleton and phosphoregulates myosin-II heavy chain, J. Cell Biol. 199 (4) (2012) 669-683.
[22] R. S. Fischer, K. A. Myers, M. L. Gardel, C. M. Waterman, Stiffness-controlled three-dimensional extracellular matrices for high-resolution imaging of cell behavior, Nat. Protoc. 7 (11) (2012) 2056-2066.
[23] J. L. Mackay, S. Kumar, Measuring the elastic properties of living cells with atomic force microscopy indentation, Methods Mol. Biol. 931 (2013) 313-329.
[24] W. Thielicke, E. J. Stamhuis, PIVlab—towards user-friendly, affordable and accurate digital particle image velocimetry in MATLAB, J. Open Res. Softw. 2 (2014).
[25] T. Das, K. Safferling, S. Rausch, N. Grabe, H. Boehm, J. P. Spatz, A molecular mechanotransduction pathway regulates collective migration of epithelial cells, Nat. Cell Biol. 17 (3) (2015) 276-287.
[26] A. J. Ridley, M. A. Schwartz, K. Burridge, R. A. Firtel, M. H. Ginsberg, G. Borisy, J. T. Parsons, A. R. Horwitz, Cell migration: integrating signals from front to back, Science 302 (5651) (2003) 1704-1709.
[27] D. T. Tambe, C. Corey Hardin, T. E. Angelini, K. Rajendran, C. Y. Park, X. Serra-Picamal, E. H. Zhou, M. H. Zaman, J. P. Butler, D. A. Weitz, J. J. Fredberg, X. Trepat, Collective cell guidance by cooperative intercellular forces, Nat. Mater. 10 (6) (2011) 469-475.
[28] C. Plutoni, E. Bazellieres, M. Le Borgne-Rochet, F. Comunale, A. Brugues, M. Seveno, D. Planchon, S. Thuault, N. Morin, S. Bodin, X. Trepat, C. Gauthier-Rouviere, P-cadherin promotes collective cell migration via a Cdc42-mediated increase in mechanical forces, J. Cell Biol. 212 (2) (2016) 199.
[29] V. A. Codelia, G. Sun, K. D. Irvine, Regulation of YAP by mechanical strain through Jnk and Hippo signaling, Curr. Biol. 24 (17) (2014) 2012-2017.
[30] M. Sun, F. Spill, M. H. Zaman, A computational model of YAP/TAZ mechanosensing, Biophysical J. 110 (11) (2016) 2540-2550.
[31] Y. Feng, L. Nie, M. D. Thakur, Q. Su, Z. Chi, Y. Zhao, G. D. Longmore, A multifunctional lentiviral-based gene knockdown with concurrent rescue that controls for off-target effects of RNAi, Genomics, Proteomics Bioinforma. 8 (4) (2010) 238-245.
[32] P. Friedl, K. Wolf, Plasticity of cell migration: a multiscale tuning model, J. Cell Biol. 188 (2010) 11-19.
[33] H. J. Lee, M. F. Diaz, K. M. Price, J. A. Ozuna, S. Zhang, E. M. Sevick-Muraca, J. P. Hagan, P. L. Wenzel, Fluid shear stress activates YAP1 to promote cancer cell motility, Nat. Commun. 8 (2017) 14122.

What is claimed is:

1. A device for evaluating cell invasion comprising:
a substrate material comprising at least two regions, wherein a first region has a first stiffness and a second region has a second stiffness; and
a plurality of cells seeded on the first region, wherein the cells are preconditioned to the first region before migrating to the second region.

2. The device of claim 1, wherein the first region mimics a primary tumor site and the second region mimics a secondary invasion site; the first region has a different stiffness value than the second region; or the first region has an increased stiffness value compared the second region.

3. The device of claim 1, wherein the substrate further comprises:
a third region comprising at least one microchannel, wherein the third region is located between the first region and the second region; or a fourth region mimicking stromal tissue, wherein the fourth region is located between the first region and the third region.

4. The device of claim 3, wherein the at least one microchannel is a flow channel.

5. The device of claim 1, wherein
the cells are mammary cells; or
the substrate comprises polyacrylamide (PA), polydimethylsiloxane (PDMS), collagen, or fibrin, or combinations thereof.

6. The device of claim 5, wherein the substrate material in the first region comprises a different polymer composition than the substrate material in the second region.

7. A method of making a device for evaluating cell invasion, the method comprising:
polymerizing a substrate comprising at least two regions, wherein a first region has a first stiffness and a second region has a second stiffness; and
seeding a plurality of cells on the first region, wherein the cells are preconditioned to the first region before migrating to the second region.

8. The method of claim 7, wherein at least a portion of the substrate is polymerized though photopolymerization.

9. The method of claim 7, wherein the substrate comprises polyacrylamide (PA), polydimethylsiloxane (PDMS), collagen, or fibrin, or combinations thereof.

10. The method of claim 7 further comprising fabricating microchannels in a third region of the substrate.

11. The method of claim 7, wherein the substrate further comprises a fourth region.

12. The method of claim 7, wherein the cells are initially limited to the first region to be preconditioned to the first region by placing a stencil over the second region to prevent migration to the second region until after the cells have been preconditioned.

13. The method of claim 7, wherein the cells are initially limited to the first region to be preconditioned to the first region by: limiting the cells seeded onto the first region, selecting a location for seeding the cells that is a distance from the second region, or increasing the first region size, or combinations thereof.

14. A method of testing a drug in vitro, comprising:
seeding cells on a first region of a device comprising a substrate comprising at least two regions, wherein the first region has a first stiffness and a second region has a second stiffness;
administering a drug to the cells on the first region or the second region; and
observing cell characteristics or observing cell migration properties.

15. The method of claim 14, wherein the observed cell characteristics or cell migration properties are selected from the group consisting of migration speed, migration distance, and molecular expressions, and combinations thereof.

16. The method of claim 14, wherein the substrate further comprises a third region comprising at least one microchannel, wherein the third region is located between the first region and the second region.

17. The method of claim 16, wherein the substrate further comprises a fourth region mimicking stromal tissue, wherein the fourth region is located between the first region and the third region.

18. The method of claim 14, wherein the cells are primary or immortalized cancer cells, optionally, squamous carcinoma, mammary cells, breast cancer cells, mixed co-cultured cell types, or primary cells from the tumor, optionally from a human or a mammal.

19. A method of identifying targets, comprising performing RNA-seq for genomic analyses to narrow down memory-related targets.

20. The method of claim 21, further comprising
disrupting a target that is identified to be implicated in memory-storing abilities; and
comparing cell characteristics or invasions after inhibiting memory-related signals.
